(19) United States
(12) Patent Application Publication
(10) Pub. No.: US 2015/0166991 A1
(43) Pub. Date: Jun. 18, 2015

(54) METHODS FOR TREATING MDS1-EVI1 MEDIATED CANCER

(71) Applicants: Archibald S. Perkins, Rochester, NY (US); Yi Zhang, Rochester, NY (US)

(72) Inventors: Archibald S. Perkins, Rochester, NY (US); Yi Zhang, Rochester, NY (US)

(73) Assignee: UNIVERSITY OF ROCHESTER, Rochester, NY (US)

(21) Appl. No.: 14/411,839

(22) PCT Filed: Jul. 1, 2013
(86) PCT No.: PCT/US13/48971

§ 371 Date: Dec. 29, 2014

Related U.S. Application Data
(60) Provisional application No. 61/666,223, filed on Jun. 29, 2012.
(60) Provisional application No. 61/738,137, filed on Dec. 17, 2012.

(51) Int. Cl. C12N 15/113; C12N 15/11; A61K 45/06; C07K 16/18; A61K 39/395; A61K 31/713

(52) U.S. Cl. CPC C12N 15/113; A61K 39/39558; A61K 31/713; A61K 45/06; C07K 16/18; C12N 15/11; C12N 2310/14

(57) ABSTRACT

The present invention relates to a method of treating a cancerous condition mediated by the protein MDS1-EVI1 (ME). The method includes administering to a patient an amount of an inhibitor of ME protein activity that is effective to cause cell death of cancer cells that are ME-dependent, thereby treating the cancerous condition. The present invention further relates to a method of causing cell death of a cancer cell that requires MDS1-EVI1 (ME) for survival. The method includes introducing an inhibitor of ME activity into a cancer cell that requires ME for survival, whereby said introducing is effective to cause cancer cell death. Novel agents that can inhibit the activity of ME in vitro or in vivo are also disclosed.

This application claims the benefit of U.S. Provisional Patent Application Ser. No. 61/666,223, filed Jun. 29, 2012, and U.S. Provisional Patent Application Ser. No. 61/738,137, filed Dec. 17, 2012, both of which are hereby incorporated by reference in their entirety.

This invention was made with government support under grant number R01CA120313 awarded by the National Institutes of Health. The government has certain rights in this invention.

### FIELD OF THE INVENTION

The present invention relates to methods and compositions for treating MDS1-EVI1 mediated cancer.

### BACKGROUND OF THE INVENTION

Leukemia is the most common form of cancer among children and adolescents (Arai et al., “Evi-1 Is a Direct Target of MLL Oncoproteins in Hematopoietic Stem Cells,” Blood (ASH Annual Meeting Abstracts) 112:3807 (November 2008)), and approximately 2,000 infants within their first year of life develop life threatening acute leukemia in the United States. The presence of the MLL (Mixed Lineage Leukemia) gene translocation is the most significant independent factor associated with poor outcome and high risk of relapse in infant leukemia (Chen et al., “Malignant Transformation Initiated by Mll-AF9: Gene Dosage and Critical Target Cells,” Cancer Cell 13:432-440 (2008); Keller and Maniatis, “Identification and Characterization of a Novel Repressor of Betainterferon Gene Expression,” Genes Dev. 5:868-879 (1991); Turner et al., “Blimp-1, a Novel Zinc Finger-Containing Protein That Can Drive the Maturation of B Lymphocytes Into Immunoglobulin-Secreting Cells,” Cell 77:297-306 (1994); and Buyse et al., “The Retinoblastoma Protein Binds to RIZ, a Zinc-Finger Protein That Shares an Epitope With the Adenovirus E1A Protein,” Proc Natl Acad Sci. U.S.A. 92:4467-4471 (1995)). The MLL-AF9 translocation t(9;11)(p22;q23) is the most common, present in 70-80% of infant and nearly 60% of AML cases (Chuikov et al., “Regulation of p53 Activity Through Lysine Methylation,” Nature 432:353-360 (2004)). Treatment with chemotherapy regimens achieve infant survival rates of about 25-45%, with high relapse rates pretransplantation being a major contributor of mortality (Nanjundan et al., “Amplification of MDS1/EVI1 and EVI1 Located in the 3q26.2 Amplicon, is Associated with Favorable Patient Prognosis in Ovarian Cancer,” Cancer Res. 67:3074-3084 (2007); Nitta et al., “Oligomerization of Evi-1 Regulated by the PR Domain Contributes to Recruitment of Corepressor CtBP,” Oncogene 24:6165-6173 (2005); and Senyuk et al., “The Distal Zinc Finger Domain of AML1/MDS1/EVI1 is an Oligomerization Domain Involved in Induction of Hematopoietic Differentiation Defects in Primary Cells In Vitro,” Cancer Res. 65:7603-7611 (2005)). Thus, a need exists for more effective therapeutic regimens.

Studies have demonstrated the MLL-AF9 fusion protein activates transcription of the MECOM (MDS1-EVI1, Myelodysplastic syndrome 1-Ecotropic virus integration site 1 COMPLEX) locus, a highly conserved proto-oncogene that plays a critical role in normal hematopoiesis (Hoyt et al., “The Evi1 Proto-Oncogene is Required at Midgestation for Neural, Heart, and Paraxial Mesenchyme Development,” Mechanisms of Development 65:55-70 (1997), Goyama et al., “Evi-1 is a Critical Regulator for Hematopoietic Stem Cells and Transformed Leukemic Cells,” Cell Stem Cell 3:207-220 (2008)). ME induces oncogenic activity by deregulation of functions such as apoptosis (Dobson et al., “The mll-AF9 Gene Fusion in Mice Controls Myeloproliferation and Specifies Acute Myeloid Leukaemogenesis,” EMBO J. 18:3564-3574 (1999)) and failure of terminal myeloid maturation (Krivtsov et al., “Transformation From Committed Progenitor to Leukaemia Stem Cell Initiated by MLL-AF9,” Nature 442(7104):818-22 (2006); and Somervaille et al., “Identification and Characterization of Leukemia Stem Cells in Murine MLL-AF9 Acute Myeloid Leukemia,” Cancer Cell 10:257-268 (2006)).

AMLs bearing MLL fusion proteins (MFPs) have a poor prognosis; chemotherapy is inadequate, indicating the need for more effective therapies (Frankel et al., “Therapeutic Trial for Infant Acute Lymphoblastic Leukemia: The Pediatric Oncology Group Experience (POG 8493),” J. Pediatr. Hematol. Oncol. 19(1):35-42 (1997); Silverman et al., “Intensified Therapy for Infants With Acute Lymphoblastic Leukemia: Results From the Dana-Farber Cancer Institute Consortium,” Cancer 80(12):2285-2295 (1997); and Reaman et al., “Treatment Outcome and Prognostic Factors for Infants with Acute Lymphoblastic Leukemia Treated on Two Consecutive Trials of the Children's Cancer Group,” J. Clin. Oncol. 17(2):445-455 (1999)). Insights into the molecular pathogenesis of ME-mediated leukemias will greatly facilitate the development of targeted agents to treat these leukemias.

Therefore, it would be desirable to determine how to target ME to treat these types of leukemias, as well as identify agents that can be used to disrupt ME activity in these and other forms of ME-dependent cancer. The present invention is directed to overcoming these and other deficiencies in the art.

### SUMMARY OF THE INVENTION

A first aspect of the invention relates to a method of treating a cancerous condition mediated by the protein MDS1-EVI1 (ME). The method includes administering to a patient an amount of an inhibitor of ME protein activity that is effective to cause cell death of cancer cells that are ME-dependent, thereby treating the cancerous condition.

A second aspect of the invention relates to a method of causing cell death of a cancer cell that requires MDS1-EVI1 (ME) for survival. The method includes introducing an inhibitor of ME activity into a cancer cell that requires ME for survival, whereby said introducing is effective to cause cancer cell death.

A third aspect of the invention relates to an inhibitor of ME protein activity selected from the group consisting of an antibody or antibody fragment that binds specifically to a PR domain of the ME protein, an anti-ME nucleic acid aptamer, a dominant negative ME fragment, or an inhibitory nucleic acid molecule that interferes with ME expression to cause a reduction in ME activity. Pharmaceutical compositions containing one or more of these inhibitors of ME protein activity, and their use in accordance with the first and second aspects of the invention, are also contemplated.

In a subset of MFP AMLs, specifically those lacking monocytic features (Bindels et al., “EVI1 is Critical for the Pathogenesis of a Subset of MLL-AF9-rearranged AMLs,” Blood 119(24):5838-5849 (2012), which is hereby incorporated by reference in its entirety), MFPs can bind to and activate transcription of MECOM (Bindels et al., EVI1 is Critical for the Pathogenesis of a Subset of MLL-AF9-rearranged AMLs,” Blood 119(24):5838-5849 (2012); Arai et al., “Evi-1 is a Transcriptional Target of Mixed-Lineage Leukemia Oncoproteins in Hematopoietic Stem Cells,” Blood 117(23):6304-6314 (2011); Chen et al., “Malignant Transformation Initiated by Mll-AF9: Gene Dosage and Critical Target Cells,” Cancer Cell 13(5):432-440 (2008), all of which are hereby incorporated by reference in their entirety), a highly conserved proto-oncogene encoding MDS1-EVI1 (ME), and EVI1 isoforms via distinct transcription sites. Relative to EVI1 ME possesses a “PR” domain with histone methyltransferase activity (Pinheiro et al., “Prdm3 and Prdm16 are H3K9me1 Methyltransferases Required for Mammalian Heterochromatin Integrity,” Cell 150(5):948-960 (2012), which is hereby incorporated by reference in its entirety). In this invention, using mouse alleles where ME is constitutively (ME^{m1}) or conditionally (ME^{fl4}) lost (Zhang et al., “PR Domain—Containing Mds1-Evi1 is Critical for Long-Term Hematopoietic Stem Cell Function,” Blood 118(14):3853-3861 (2011), which is hereby incorporated by reference in its entirety), it is revealed that the PR domain is essential in MFP transformation in mouse. These results implicate ME as a novel target for therapeutic intervention.

A subgroup of leukemogenic MLL fusion proteins (MFP) including MLL-AF9 activates Mecom locus, and exhibits extremely poor clinical prognosis. MECOM encodes EVI1 and MDS1-EVI1 (ME) proteins via alternative transcription start sites; these differ by the presence of a SET-like PR domain at the N-terminus of ME. SET domains are known to have histone methyltransferase (HMT) activity, and to play important roles in chromatin modification and the regulation of gene expression. The function of the PR domain of ME is unknown. The ability of ME-deficient Lin−/Sca1+/c-Kit+ (LSK) cells to be transformed by different leukemogenic oncogenes were tested. This revealed that while NUP98-HOXA9, E2A-HLF, and MEIS1-HOXA9 were able to transform ME-deficient cells, both MLL-AF9 and MLL-ENL (MLL Fusion Proteins, (MFP)) were ineffective, indicating an essential function for ME in the context of MFP-induced acute myeloid leukemia (AML). Experiments with a conditional allele of ME show that the gene is important not only for the initiation but also the continued survival of MFP AML cells. Structure-function studies demonstrated that within ME, the PR domain is essential for MFP-induced AML. In silico analysis of the PR domain indicates strong similarity to the structure of SET domains with known HMT activity: a series of β-sheet structures with a substrate lysine binding pocket stabilized by a salt bridge between D₁₃₀ and R₁₇₆, and with a conserved Y (111) residue at the catalytic site. This in silico analysis also revealed important differences, including the absence of the i-SET domain, which is known to provide contacts for binding of substrate histone and methyl donor, S-adenosylmethionine (SAM). In its place were W(149) and Q(115) residues, pointing towards the exterior; the placement of these residues indicates a role in the docking of another protein that serves as the i-SET domain. Finally, there is a change of a Y involved in catalysis to M (residue 191 in ME). To test if the PR domain of ME requires these putative structures for activity, three residues were mutated to A: Y₁₁₁, R₁₇₆, and W₁₄₉. All three mutations resulted in loss of rescue activity, while a control mutation away from the pocket/catalytic site (C158A) had no effect. These data support the contention that (1) ME is essential for MFP-induced AML; (2) this activity depends on the PR domain; and (3) within the PR domain, critical structures include a putative substrate binding pocket, catalytic Y residue, and docking site for an interacting protein. Importantly, these findings strongly indicate that it should be possible to inhibit the function of the PR domain of ME, and that such inhibition should result in the death of MFP-induced leukemias. These studies clearly indicate an essential role of PR-domain protein ME in MFP leukemia, in which case ME represents a novel target for therapeutic intervention for this group of leukemias.

### BRIEF DESCRIPTION OF THE DRAWINGS

FIGS. 1A-F illustrate that ME^{m1/m1} bone marrow is resistant to MLL-AF9-induced transformation. FIG. 1A is a diagram of the ME^{m1} allele, showing exon 1 of Mds1, with putative transcription start site and splice donor site, as well as site of lacZ insertion and the extent of the DNA of the first intron deleted. Also shown are the locations of MFP binding described by Arai et al., “Evi-1 is a Transcriptional Target of Mixed-Lineage Leukemia Oncoproteins in Hematopoietic Stem Cells,” Blood 117(23):6304-6314 (2011), which is hereby incorporated by reference in its entirety), as well as the MFP-responsive region identified by this same group by luciferase reporter assays (Arai et al., “Evi-1 is a Transcriptional Target of Mixed-Lineage Leukemia Oncoproteins in Hematopoietic Stem Cells,” Blood 117(23):6304-6314 (2011), which is hereby incorporated by reference in its entirety). FIG. 1B shows quantitation of the number of colonies formed in growth factor-supplemented methylcellulose at each of four replatings, for wildtype (WT) and ME^{m1/m1} bone marrow. Error bars represent standard deviation of platings done in triplicate, 1000 cells/plate. The experiment was repeated multiple times with the same result. FIGS. 1C, 1D, and 1E show results of ME requirement being restricted to transformation by MLL fusion genes. In FIG. 1C, quantitation of the number of colonies formed in growth factor-supplemented methylcellulose at each of four replatings for ME^{m1/m1} LSK cells transduced with the virus indicated is shown. Error bars are standard deviation; p values determined by Student T test, comparing the first and fourth plating. FIGS. 1D and 1E show serial replating assay of bone marrow from mice of the genotype indicated, transduced with the leukemogenic oncogene indicated, and treated or not with 4-hydroxy tamoxifen (4-OH-TAM) (1 μM) as indicated. Error bars are not shown, to avoid an overly busy figure, but are within 10% of the value of each bar; p values were calculated by Student T test, comparing first and fourth plating. In FIG. 1F, ME but not EVI1 or MDS1 can rescue the transformation deficiency of ME^{m1/m1} bone marrow. Serial replating transformation assay of LSK cells isolated from either WT or ME^{m1/m1} bone marrow, transduced with MLL-AF9 as well as retroviral expression construct indicated: MIGR1 (empty vector), MDS1, EVI1 or ME. Error bars denote standard deviation. A third replating yielded no colonies for MIGR1 and EVI1-transduced cells.

FIGS. 2A-H illustrate that deletion of ME results in failure of MLL-AF9 leukemic cells to transplant into syngeneic sublethally irradiated recipient mice. FIG. 2A is a diagram that depicts experimental procedures. FIGS. 2B, 2C, and 2D as shown are complete blood count data from weeks 0 to 4 post-transplant for recipients of ME^{fl1/m1} cells, with and without pre-treatment with 4-OH TAM; extent of normal values is indicated by greyed zone. Mice receiving cells with no 4-OH TAM pre-treatment became frankly leukemic, anemic, and thrombocytopenic over the four weeks of monitoring. At week four, most recipients of the untreated cells were moribund, and the mice were necropsied. Error bars depict standard deviation. Statistical significance (Student T test) was observed at week four for leukocytes and platelets (p<0.05). FIGS. 2E, 2F, 2G, and 2H show that deletion of ME results in failure of leukemic cells to significantly infiltrate organs of irradiated recipients. Mice receiving 4-OH TAM-pretreated MLL-AF9 leukemia cells maintained normal spleen weights (FIGS. 2E and 2F), as well as livers and bone marrow essentially devoid of infiltrating leukemia. Non-pretreated cells infiltrated spleen, liver, and bone marrow. In FIG. 2E, gross photographs of spleens from mice injected with un-treated and 4-OH TAM pre-treated cells are shown. FIG. 2F shows scattergrams of spleen weights of the four experimental groups, as indicated; the number of spleens in each cohort is in parentheses. The average is demarcated by horizontal bar; and p value determined by Student T test. FIGS. 2G and 2H show photomicrographs of liver (FIG. 2G) and bone marrow (FIG. 2H), showing extensive infiltration by leukemic cells. Photographs are at 200× magnification with H&E staining.

FIGS. 3A-C show that the PR domain conforms in structure to enzymatically active SET domains, albeit with important differences. FIG. 3A is a partial alignment of PR domain of murine ME (SEQ ID NO: 15) with the SET domains of human Suvar39H1 (SEQ ID NO: 16) and P. sativa RubiscoLSMT (SEQ ID NO: 17), with demarcation of the regions of beta sheet 1 through 12 and location of the iSET domain. The numbering above refers to ME, below to RuLSMT. FIGS. 3B-C illustrate the ribbon structure of the lysine binding pocket from RubiscoLSMT and ME, respectively, with key residues underlined. The diagonal line denotes actual (FIG. 3B) and potential (FIG. 3C) interaction between residues as discussed in the examples. Beta sheets 6, 7, 9, and 12 are indicated; P6 is perpendicular to the viewing plane.

FIGS. 4A-E illustrate that Y₁₁₁, W₁₄₉, and R₁₇₆ of PR domain of ME are required for biologic activity. FIG. 4A shows the results of a replating assay performed with lineage-negative cells of ME^{m1/m1} mice. Shown is a photograph of the third plating, in duplicate. FIG. 4B is a backbone depiction of in silico structure of PR protein, showing locations of the amino acids mutated. FIG. 4C shows a higher magnification view of “substrate pocket” of PR domain, showing location of the W149, protruding from the pocket, as well as the Y111 and R176. FIG. 4D is an alignment showing the conservation of Y111 among enzymatically active SET domains, including MDS-EVI1 (amino acids 90-123 of SEQ ID NO: 1), HS Suvar39H1 (amino acids 12-44 of SEQ ID NO: 16), EZH2 (SEQ ID NO: 18), EZH1 (SEQ ID NO: 19), E(Z) (SEQ ID NO: 20), and MLL (SEQ ID NO: 21). FIG. 4E is a Western blot of transfected HEK293T cells showing expression of mutant or wildtype ME, at the expected molecular weight.

FIG. 5 illustrates the evolutionary relationship of PR/SET proteins. (Figure is adapted from Wu et al., “Structural Biology of Human H3K9 Methyltransferases,” PLoS One 1:e8570 (2010), which is hereby incorporated by reference in its entirety.)

FIGS. 6A-B illustrate the structure of the Mecom genomic locus (FIG. 6A) and the structure of the Mecom proteins (FIG. 6B). In FIG. 6A, the genomic structure of the Mecom locus is illustrated, showing starts of transcription at Mds1 exon 1 (Mds1ex1) and Evi1, as well as mRNA splicing pattern. Scale is in basepairs. Below is an enlargement of the Evi1 portion of the locus, showing Evi1 exons 1-15, as well as locations of the genetic lesions that have been reported: floxed exon 3 (Zhang et al., “PR-Domain—Containing Mds1-Evi1 is Critical for Long-Term Hematopoietic Stem Cell Function,” Blood 118:3853 (2011), which is hereby incorporated by reference in its entirety); floxed exon 4 (Goyama et al., “Evi-1 is a Critical Regulator for Hematopoietic Stem Cells and Transformed Leukemic Cells,” Cell Stem Cell 3:207 (2008), which is hereby incorporated by reference in its entirety); and Neo gene insertion into exon 7 (Hoyt et al., “The Evi1 Proto-Oncogene is Required at Midgestation for Neural, Heart, and Paraxial Mesenchyme Development,” Mechanisms of Development 65:55 (1997), which is hereby incorporated by reference in its entirety). Also indicated are the presence of initiator methionines in exons 3 and 4. In FIG. 6B, the structure of the proteins derived from the Mecom locus are shown, with the apparent molecular weights indicated to the left, as well as key structural elements, including the PR domain, the zinc fingers (ZF), the C-terminal binding protein (CtBP).

FIG. 7 illustrates one example of a chimeric therapeutic agent that includes an AML-specific aptamer KH1C12 and either an ME-inhibiting aptamer or ME-inhibiting RNAi molecule. The therapeutic agent is targeted to AML cells recognized by the KH1C12 aptamer.

FIG. 8 illustrates an approach for targeted delivery of a therapeutic agent with a conjugated aptamer molecule. One or more AML-specific KH1C12 aptamers and one or more ME-inhibiting aptamers or ME-inhibiting RNAi molecules form the functional components of the conjugate. The therapeutic agent is targeted to an AML cell recognized by the KH1C12 aptamer.

FIG. 9 shows a targeted approach for delivery of a therapeutic agent having a conjugate that includes a polycation-aptamer or RNAi vector linked via phenyl(di)boronic acid-salicylhydroxamic acid assembly to an antibody that is specific for a cancer cell surface marker (e.g., CD-33, CD-19, or CD-20). The therapeutic agent is targeted to cells that express the cancer cell surface marker.

### DETAILED DESCRIPTION OF THE INVENTION

One aspect of the invention relates to a method of treating a cancerous condition mediated by the protein MDS1-EVI1 (“ME”). The method includes administering to a patient an amount of an inhibitor of ME protein activity that is effective to cause cell death of cancer cells that are ME-dependent, thereby treating the cancerous condition.

A related aspect of the invention relates to a method of causing death of a cancer cell that requires ME for survival. This method includes introducing an inhibitor of ME activity into a cancer cell that requires ME for survival under conditions effective to cause cancer cell death.

Messenger RNA transcripts initiating at Mds1 can splice from exon 2 of Mds1 into exon 2 of Evi1 to encode a larger ME protein. Relative to EVI1 proper, the ME protein possesses a 190 amino acid N-terminal extension encoded by exon 2 of Mds1 and exon 2 of Evi1. The latter is all open reading frame, in frame with the coding of exon 3 of Evi1, but cannot be translated in the context of an Evi1 transcript due to the lack of an initiator methionine (the first of which resides in exon 3 of Evi1). However, within the context of Mds1-Evi1 mRNA transcripts, exon 2 is translated, and, together with exon 2 of Mds1, encodes a 110 amino acid domain with homology to a domain present in other proteins, specifically positive regulatory domain 1 binding factor 1 (PRD1-BF1) (Keller and Maniatis, “Identification and Characterization of a Novel Repressor of Betainterferon Gene Expression,” Genes Dev. 5:868-879 (1991), which is hereby incorporated by reference in its entirety) (also known as B-lymphocyte-induced maturation protein 1 (Blimp-1) (Turner et al., “Blimp-1, a Novel Zinc Finger-Containing Protein That can Drive the Maturation of B Lymphocytes into Immunoglobulin-Secreting Cells,” Cell 77:297-306 (1994), which is hereby incorporated by reference in its entirety) and Rb-interacting zinc finger protein (RIZ) (Buyse et al., “The Retinoblastoma Protein Binds to RIZ, a Zinc-Finger Protein That Shares an Epitope With the Adenovirus ElA Protein,” Proc. Natl. Acad. Sci. USA 92:4467-4471 (1995), which is hereby incorporated by reference in its entirety). This domain, termed the PRD1-BF1-RIZ (PR) domain is evolutionarily distantly related to the SET domain (Su(var)3-9, Enhancer of zeste (E(z)), and Trithorax (trx).

A number of types of cancer are associated with ME expression and are believed to be ME-dependent, i.e., requiring ME expression and activity for either cancer cell development, cancer cell survival, or both. Exemplary types of cancer or precancerous conditions that are associated with ME expression include, without limitation, cancer cells that are leukemic or dysplastic such as acute myeloid leukemia (AML), acute lymphoid leukemia (ALL), myelodysplastic syndrome (MDS), chronic myelogenous leukemia (CML), and epithelial cancers where the 3q26.2 aberration is present (ovary, breast, head and neck, cervix, and lung). In certain AML or ALL cancers, the cancerous condition is associated with the presence of the MLL-AF9 translocation t(9;11)(p22;q23) or MLL-ENL translocation t(11;19)(q23;p13.3); these translocations encode MLL fusion proteins, and some of these have been found to activate transcription of Mecom and MLL fusion protein induced leukemias have been found to depend on an intact Mecom gene (Goyama et al., “Evi-1 is a Critical Regulator for Hematopoietic Stem Cells and Transformed Leukemic Cells,” Cell Stem Cell 3:207-220 (2008), which is hereby incorporated by reference in its entirety).

The human ME amino acid sequence, variant c, is described at Genbank Accession NP_{—}004982.2 and comprises the amino acid sequence set forth below (SEQ ID NO: 1):

MRSKGRARKL ATNNECVYGN YPEIPLEEMP DADGVASTPS
LNIQEPCSPA TSSEAFTPKE GSPYKAPIYI PDDIPIPAEF
ELRESNMPGA GLGIWTKRKI EVGEKFGPYV GEQRSNLKDP
SYGWEILDEF YNVKFCIDAS QPDVGSWLKY IRFAGCYDQH
NLVACQINDQ IFYRVVADIA PGEELLLFMK SEDYPHETMA
PDIHEERQYR CEDCDQLFES KAELADHQKF PCSTPHSAFS
MVEEDFQQKL ESENDLQEIH TIQECKECDQ VFPDLQSLEK
HMLSHTEERE YKCDQCPKAF NWKSNLIRHQ MSHDSGKHYE
CENCAKVFTD PSNLQRHIRS QHVGARAHAC PECGKTFATS
SGLKQHKHIH SSVKPFICEV CHKSYTQFSN LCRHKRMHAD
CRTQIKCKDC GQMFSTTSSL NKHRRFCEGK NHFAAGGFFG
QGISLPGTPA MDKTSMVNMS HANPGLADYF GANRHPAGLT
FPTAPGFSFS FPGLFPSGLY HRPPLIPASS PVKGLSSTEQ
TNKSQSPLMT HPQILPATQD ILKALSKHPS VGDNKPVELQ
PERSSEERPF EKISDQSESS DLDDVSTPSG SDLETTSGSD
LESDIESDKE KFKENGKMFK DKVSPLQNLA SINNKKEYSN
HSIFSPSLEE QTAVSGAVND SIKAIASIAE KYFGSTGLVG
LQDKKVGALP YPSMFPLPFF PAFSQSMYPF PDRDLRSLPL
KMEPQSPGEV KKLQKGSSES PFDLTTKRKD EKPLTPVPSK
PPVTPATSQD QPLDLSMGSR SRASGTKLTE PRKNHVFGGK
KGSNVESRPA SDGSLQHARP TPFFMDPIYR VEKRKLTDPL
EALKEKYLRP SPGFLFHPQF QLPDQRTWMS AIENMAEKLE
SFSALKPEAS ELLQSVPSMF NFRAPPNALP ENLLRKGKER
YTCRYCGKIF PRSANLTRHL RTHTGEQPYR CKYCDRSFSI
SSNLQRHVRN IHNKEKPFKC HLCDRCFGQQ TNLDRHLKKH
ENGNMSGTAT SSPHSELEST GAILDDKEDA YFTEIRNFIG
NSNHGSQSPR NVEERMNGSH FKDEKALVTS QNSDLLDDEE
VEDEVLLDEE DEDNDITGKT GKEPVTSNLH EGNPEDDYEE
TSALEMSCKT SPVRYKEEEY KSGLSALDHI RHFTDSLKMR
KMEDNQYSEA ELSSFSTSHV PEELKQPLHR KSKSQAYAMM
LSLSDKESLH STSHSSSNVW HSMARAAAES SAIQSISHV

The PR domain is underlined in the sequence shown above, and residues Y₁₀₉, R₁₇₄, and W₁₄₇ critical to PR domain (and ME protein) function are shown in bold typeface. The nucleotide sequence encoding this ME protein is provided below and reported at Genbank Accession NM_{—}004991.3 as follows (SEQ ID NO: 2):

atgagatccaaaggcagggcaaggaaactggccacaaataatgagtgtgt
atatggcaactaccctgaaatacctttggaagaaatgccagatgcagatg
gagtagccagcactccctccctcaatattcaagagccatgctctcctgcc
acatccagtgaagcattcactccaaaggagggttctccttacaaagcccc
catctacatccctgatgatatccccattcctgctgagtttgaacttcgag
agtcaaatatgcctggggcaggactaggaatatggaccaaaaggaagatc
gaagtaggtgaaaagtttgggccttatgtgggagagcagaggtcaaacct
gaaagaccccagttatggatgggagatcttagacgaattttacaatgtga
agttctgcatagatgccagtcaaccagatgttggaagctggctcaagtac
attagattcgctggctgttatgatcagcacaaccttgttgcatgccagat
aaatgatcagatattctatagagtagttgcagacattgcgccgggagagg
agcttctgctgttcatgaagagcgaagactatccccatgaaactatggcg
ccggatatccacgaagaacggcaatatcgctgcgaagactgtgaccagct
ctttgaatctaaggctgaactagcagatcaccaaaagtttccatgcagta
ctcctcactcagcattttcaatggttgaagaggactttcagcaaaaactc
gaaagcgagaatgatctccaagagatacacacgatccaggagtgtaagga
atgtgaccaagtttttcctgatttgcaaagcctggagaaacacatgctgt
cacatactgaagagagggaatacaagtgtgatcagtgtcccaaggcattt
aactggaagtccaatttaattcgccaccagatgtcacatgacagtggaaa
gcactatgaatgtgaaaactgtgccaaggttttcacggaccctagcaacc
ttcagcggcacattcgctctcagcatgtcggtgcccgggcccatgcatgc
ccggagtgtggcaaaacgtttgccacttcgtcgggcctcaaacaacacaa
gcacatccacagcagtgtgaagccctttatctgtgaggtctgccataaat
cctatactcagttttcaaacctttgccgtcataagcgcatgcatgctgat
tgcagaacccaaatcaagtgcaaagactgtggacaaatgttcagcactac
gtcttccttaaataaacacaggaggttttgtgagggcaagaaccattttg
cggcaggtggattttttggccaaggcatttcacttcctggaaccccagct
atggataaaacgtccatggttaatatgagtcatgccaacccgggccttgc
tgactattttggcgccaataggcatcctgctggtcttacctttccaacag
ctcctggattttcttttagcttccctggtctgtttccttccggcttgtac
cacaggcctcctttgatacctgctagttctcctgttaaaggactatcaag
tactgaacagacaaacaaaagtcaaagtcccctcatgacacatcctcaga
tactgccagctacacaggatattttgaaggcactatctaaacacccatct
gtaggggacaataagccagtggagctccagcccgagaggtcctctgaaga
gaggccctttgagaaaatcagtgaccagtcagagagtagtgaccttgatg
atgtcagtacaccaagtggcagtgacctggaaacaacctcgggctctgat
ctggaaagtgacattgaaagtgataaagagaaatttaaagaaaatggtaa
aatgttcaaagacaaagtaagccctcttcagaatctggcttcaataaata
ataagaaagaatacagcaatcattccattttctcaccatctttagaggag
cagactgcggtgtcaggagctgtgaatgattctataaaggctattgcttc
tattgctgaaaaatactttggttcaacaggactggtggggctgcaagaca
aaaaagttggagctttaccttacccttccatgtttcccctcccatttttt
ccagcattctctcaatcaatgtacccatttcctgatagagacttgagatc
gttacctttgaaaatggaaccccaatcaccaggtgaagtaaagaaactgc
agaagggcagctctgagtccccctttgatctcaccactaagcgaaaggat
gagaagcccttgactccagtcccctccaagcctccagtgacacctgccac
aagccaagaccagcccctggatctaagtatgggcagtaggagtagagcca
gtgggacaaagctgactgagcctcgaaaaaaccacgtgtttgggggaaaa
aaaggaagcaacgtcgaatcaagacctgcttcagatggttccttgcagca
tgcaagacccactcctttctttatggaccctatttacagagtagagaaaa
gaaaactaactgacccacttgaagctttaaaagagaaatacttgaggcct
tctccaggattcttgtttcacccacaattccaactgcctgatcagagaac
ttggatgtcagctattgaaaacatggcagaaaagctagagagcttcagtg
ccctgaaacctgaggccagtgagctcttacagtcagtgccctctatgttc
aacttcagggcgcctoccaatgccctgccagagaaccttctgcggaaggg
aaaggagcgctatacctgcagatactgtggcaagatttttccaaggtctg
caaacctaacacggcacttgagaacccacacaggagagcagccttacaga
tgcaaatactgtgacagatcatttagcatatcttctaacttgcaaaggca
tgttcgcaacatccacaataaagagaagccatttaagtgtcacttatgtg
ataggtgttttggtcaacaaaccaatttagacagacacctaaagaaacat
gagaatgggaacatgtccggtacagcaacatcgtcgcctcattctgaact
ggaaagtacaggtgcgattctggatgacaaagaagatgcttacttcacag
aaattcgaaatttcattgggaacagcaaccatggcagccaatctcccagg
aatgtggaggagagaatgaatggcagtcattttaaagatgaaaaggcttt
ggtgaccagtcaaaattcagacttgctggatgatgaagaagttgaagatg
aggtgttgttagatgaggaggatgaagacaatgatattactggaaaaaca
ggaaaggaaccagtgacaagtaatttacatgaaggaaaccctgaggatga
ctatgaagaaaccagtgccctggagatgagttgcaagacatccccagtga
ggtataaagaggaagaatataaaagtggactttctgctctagatcatata
aggcacttcacagatagcctcaaaatgaggaaaatggaagataatcaata
ttctgaagctgagctgtcttcttttagtacttcccatgtgccagaggaac
ttaagcagccgttacacagaaagtccaaatcgcaggcatatgctatgatg
ctgtcactgtctgacaaggagtccctccattctacatcccacagttcttc
caacgtgtggcacagtatggccagggctgcggcggaatccagtgctatcc
agtccataagccacgtatga

An exemplary mouse ME amino acid sequence is described partially by Swissprot accession number G3UWT0 and comprises the amino acid sequence set forth below (SEQ ID NO: 3):

MRSKGRARKL ATSNECAYGN YPEIPLEEMP DADADGITSV
PSLHIQEPCS PATSSESFTP KEGSPYKAPI YIPDDIPIPD
EFELRESTMP GAGLGIWTKR KIEIGEKFGP YMGEQRSDLK
DSSYGWEILD EFCNVKFCID ASQPDVGSWL KYIRFAGCYD
QHNLVACQIN DQIFYRVVAD IAPGEELLLF MKSEEDPHEP
MAPDIHEERQ HRCEDCDQLF ESKAELADHQ KFPCSTPHSA
FSMVEEDLQQ NLESESDLRE IHGNQDCKEC DRVFPDLQSL
EKHMLSHTEE REYKCDQCPK AFNWKSNLIR HQMSHDSGKH
YECENCAKVF TDPSNLQRHI RSQHVGARAH ACPECGKTFA
TSSGLKQHKH IHSSVKPFIC EVCHKSYTQF SNLCRHKRMH
ADCRTQIKCK DCGQMFSTTS SLNKHRRFCE GKNHFAAGGF
FGQGISLPGT PAMDKTSMVN MSHANPGLAD YFGTNRHPAG
LTFPTAPGFS FSFPGLFPSG LYHRPPLIPA SPPVKGLSST
EQSNKCQSPL LTHPQILPAT QDILKALSKH PPVGDNKPVE
LLPERSSEER PLEKISDQSE SSDLDDVSTP SGSDLETTSG
SDLESDLESD KEKCKENGKM FKDKVSPLQN LASITNKKEH
NNHSVFSASV EEQSAVSGAV NDSIKAIASI AEKYFGSTGL
VGLQDKKVGA LPYPSMFPLP FFPAFSQSMY PFPDRDLRSL
PLKMEPQSPS EVKKLQKGSS ESPFDLTTKR KDEKPLTSGP
SKPSGTPATS QDQPLDLSMG SRGRASGTKL TEPRKNHVFG
EKKGSNMDTR PSSDGSLQHA RPTPFFMDPI YRVEKRKLTD
PLEALKEKYL RPSPGFLFHP QMSAIENMAE KLESFSALKP
EASELLQSVP SMFSFRAPPN TLPENLLRKG KERYTCRYCG
KIFPRSANLT RHLRTHTGEQ PYRCKYCDRS FSISSNLQRH
VRNIHNKEKP FKCHLCDRCF GQQTNLDRHL KKHENGNMSG
TATSSPHSEL ESAGAILDDK EDAYFTEIRN FIGNSNHGSQ
SPRNMEERMN GSHFKDKKAL ATSQNSDLLD DEEVEDEVLL
DEEDEDNDIP GKPRKELGVT RLDEEIPEDD YEEAGALEMS
CKASPVRYKE EDYKSGLSAL DHIRHFTDSL KMREMEENQY
TDAELSSISS SHVPEELKQT LHRKSKSQAY AMMLSLSDKD
SLHPTSHSSS NVWHSMARAA AESSAIQSIS HV

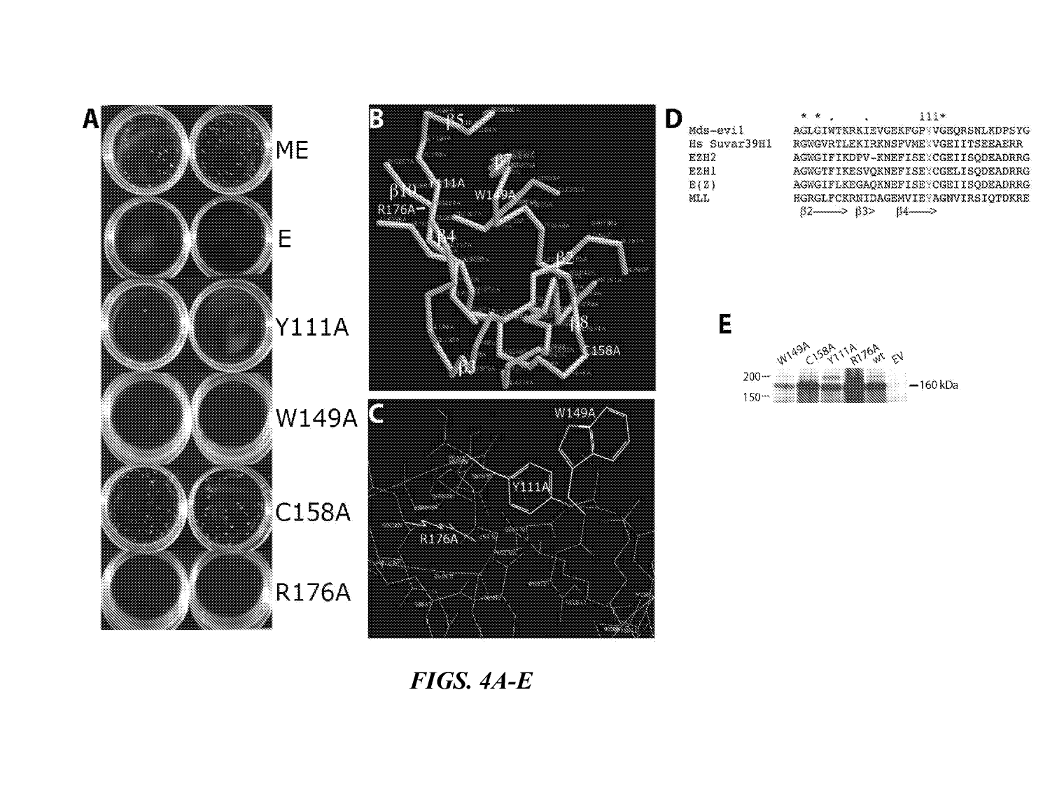

The PR domain is underlined in the sequence shown above. This protein sequence is further analyzed in the Examples section, specifically in association with FIGS. 4A-E. The nucleotide sequence encoding this ME protein is provided below and reported at Genbank Accession CN697723.1 plus M21829. CN697723 contains a partial sequence that bridges between MDS1 and EVI1 (M21829), and the remainder of the open reading frame is afforded by the remainder of EVI1 (SEQ ID NO: 4):

ATGAGATCCAAAGGCAGGGCAAGGAAACTGGCCACAAGTAATGAGTGTGC
CTATGGCAACTATCCTGAAATACCTTTGGAAGAAATGCCAGATGCTGATG
CAGATGGGATAACCAGTGTTCCCTCCCTCCACATTCAAGAGCCATGCTCT
CCTGCGACGTCCAGTGAGTCATTTACTCCTAAGGAGGGCTCGCCATACAA
AGCTCCCATCTACATCCCTGATGACATCCCTATTCCTGATGAGTTTGAGC
TTCGAGAGTCAACTATGCCTGGAGCAGGACTTGGAATATGGACCAAAAGG
AAGATTGAAATAGGCGAAAAGTTTGGGCCATACATGGGAGAGCAGAGATC
AGACCTGAAAGATTCCAGCTATGGATGGGAGATCTTAGATGAGTTTTGCA
ATGTGAAGTTCTGCATAGATGCCAGTCAACCAGATGTAGGAAGCTGGCTC
AAGTACATCAGATTCGCTGGCTGCTATGATCAGCACAACCTTGTTGCATG
CCAGATAAATGATCAGATATTCTACCGAGTAGTCGCAGACATTGCGCCTG
GGGAAGAGCTCTTGCTGTTCATGAAGAGTGAAGAGGACCCGCACGAACCC
ATGGCGCCTGACATCCACGAAGAACGGCAGCACCGCTGTGAGGACTGTGA
CCAGCTCTTTGAATCCAAGGCAGAGCTAGCCGATCACCAGAAGTTCCCAT
GCAGCACACCTCACTCGGCCTTCTCCATGGTGGAGGAGGACTTGCAACAA
AACCTGGAGAGTGAGAGCGATCTCCGAGAGATCCATGGCAACCAGGACTG
TAAGGAATGTGACCGAGTTTTCCCCGATCTGCAAAGCTTGGAGAAGCACA
TGCTGTCACATACTGAGGAGAGGGAATACAAGTGTGATCAGTGTCCCAAG
GCATTTAACTGGAAGTCCAATTTAATTCGCCACCAGATGTCACATGACAG
TGGAAAGCACTATGAGTGTGAAAACTGTGCCAAGGTTTTCACGGACCCTA
GCAACCTTCAGCGACACATTCGATCTCAGCATGTTGGTGCCCGGGCTCAT
GCTTGCCCCGAGTGTGGTAAAACATTTGCCACTTCGTCAGGCCTCAAACA
GCACAAGCACATCCACAGCAGTGTGAAGCCCTTTATCTGTGAGGTCTGCC
ATAAATCCTATACTCAGTTTTCAAACCTTTGTCGTCATAAGCGCATGCAT
GCTGATTGCAGAACCCAAATCAAGTGCAAAGACTGTGGACAAATGTTCAG
CACTACGTCTTCCTTAAATAAACACAGGAGGTTTTGTGAGGGCAAGAACC
ATTTTGCGGCAGGTGGATTTTTTGGCCAAGGCATTTCACTTCCTGGAACC
CCAGCTATGGATAAAACGTCCATGGTTAATATGAGTCATGCCAACCCGGG
CCTGGCTGACTATTTTGGCACCAATAGGCATCCTGCTGGTCTTACCTTTC
CAACAGCTCCTGGATTTTCCTTTAGCTTTCCTGGTCTGTTTCCTTCGGGC
TTATACCACAGGCCTCCTTTGATACCCGCTAGTCCTCCTGTTAAAGGATT
ATCAAGTACCGAACAGTCAAACAAATGTCAAAGTCCCCTCTTGACACATC
CTCAGATACTGCCAGCTACACAGGATATTTTGAAGGCACTATCTAAACAC
CCACCTGTAGGGGACAATAAGCCAGTGGAACTGCTGCCCGAGAGGTCCTC
TGAAGAGAGGCCCCTTGAGAAAATCAGTGACCAGTCAGAGAGTAGTGACC
TTGATGATGTCAGTACACCAAGTGGCAGTGACCTGGAAACAACCTCGGGC
TCTGATCTGGAAAGTGACCTTGAAAGTGATAAAGAGAAATGTAAAGAAAA
TGGTAAAATGTTCAAAGACAAAGTAAGCCCTCTGCAGAATCTGGCTTCGA
TAACTAATAAGAAAGAACACAACAATCATTCCGTTTTCTCAGCATCTGTA
GAGGAGCAAAGTGCCGTGTCAGGAGCTGTGAATGATTCTATAAAGGCTAT
TGCGTCTATTGCTGAAAAATACTTTGGTTCTACAGGACTGGTGGGGCTGC
AAGACAAAAAAGTTGGAGCGTTACCTTACCCTICCATGTTTCCCCTCCCG
TTTTTTCCAGCATTCTCTCAATCAATGTACCCATTTCCTGATAGAGACTT
GAGGTCGTTACCTTTGAAAATGGAGCCCCAATCACCAAGTGAAGTTAAGA
AACTGCAGAAGGGAAGCTCTGAGTCCCCTTTTGACCTCACCACTAAGAGA
AAGGATGAGAAGCCCTTGACTTCAGGCCCCTCGAAGCCTTCAGGAACACC
AGCCACAAGCCAAGACCAGCCCCTGGATCTAAGTATGGGCAGTAGGGGTA
GAGCCAGTGGGACAAAGTTGACTGAGCCTCGAAAAAACCATGTGTTTGGG
GAAAAGAAAGGAAGCAACATGGATACTAGGCCATCTTCAGATGGCTCCTT
GCAGCATGCCAGACCCACTCCCTTCTTCATGGACCCCATTTATAGAGTAG
AGAAAAGAAAGTTAACTGACCCGCTTGAAGCTTTGAAAGAAAAATACTTG
AGACCTTCTCCAGGATTCTTGTTTCACCCGCAAATGTCAGCAATTGAGAA
CATGGCAGAAAAGCTGGAAAGCTTCAGCGCCCTCAAACCTGAGGCCAGCG
AGCTCCTGCAGTCCGTGCCCTCCATGTTCAGCTTCCGAGCTCCTCCCAAC
ACCCTGCCAGAGAACCTGCTGCGGAAGGGGAAAGAGCGCTACACCTGCAG
GTACTGTGGCAAGATATTTCCAAGGTCTGCGAACCTAACACGGCACTTGA
GAACCCACACAGGAGAGCAACCTTACAGATGCAAATACTGTGATAGATCA
TTCAGCATTTCTTCCAACCTGCAGCGACATGTGCGCAACATCCACAACAA
GGAGAAGCCATTTAAGTGTCATTTATGTGACAGATGTTTTGGTCAACAAA
CCAATCTTGACAGACACCTGAAGAAACATGAGAACGGCAACATGTCTGGG
ACGGCAACGTCCTCGCCTCACTCAGAGCTAGAAAGCGCAGGCGCAATCCT
GGATGACAAAGAAGATGCGTACTTTACAGAGATCCGCAATTTCATCGGGA
ACAGCAACCATGGTAGCCAGTCTCCTCGGAACATGGAAGAGAGGATGAAT
GGCAGTCACTTCAAGGATAAAAAGGCTTTGGCAACCAGCCAAAATTCAGA
TTTATTGGATGATGAAGAAGTAGAAGATGAGGTGTTGTTGGATGAGGAGG
ATGAAGACAATGATATTCCTGGAAAGCCCAGAAAGGAGCTAGGGGTGACT
CGTTTAGACGAGGAGATCCCGGAGGATGACTACGAAGAAGCTGGTGCCCT
GGAGATGAGCTGTAAGGCGTCCCCGGTGAGGTATAAAGAGGAAGACTATA
AATCTGGCCTTTCTGCTCTAGATCACATAAGGCACTTCACAGATAGCCTC
AAAATGAGGGAAATGGAAGAGAATCAATACACTGACGCTGAGCTGTCCTC
CATTAGTTCTTCTCATGTGCCAGAGGAGCTTAAACAGACGTTACACAGAA
AGTCCAAATCACAGGCATATGCTATGATGTTGTCACTGTCTGACAAGGAT
TCCCTCCATCCCACCTCCCACAGTTCCTCCAACGTGTGGCACAGCATGGC
AAGGGCTGCAGCAGAATCCAGTGCCATCCAGTCCATAAGCCATGTATGA

A ClustalW alignment of the human and mouse ME PR domains (amino acids 84-193 of SEQ ID NOs: 1, 3) is provided below:

| | |
|---|---|
| | 111 115 130 |
| MousePR | LRESTMPGAGLGIWTKRKIEIGEKFGPYMGEQRSDLKDSSYGWEILDEFCNVKFCIDASQ |
| HumanPR | LRESNMPGAGLGIWTKRKIEVGEKFGPYVGEQRSNLKDPSYGWEILDEFYNVKFCIDASQ |
| | ****.***************:*******:*****:***.********** ********** |
| | 149 176 191 |
| MousePR | PDVGSWLKYIRFAGCYDQHNLVACQINDQIFYRVVADIAPGEELLLFMKS |
| HumanPR | PDVGSWLKYIRFAGCYDQHNLVACQINDQIFYRVVADIAPGEELLLFMKS |
| | ************************************************** |

where the symbol (:) indicates that the amino acids variations share strongly similar properties, scoring >0.5 in the Gonnet PAM 250 matrix; the symbol (.) indicates that the amino acid variations share weakly similar properties, scoring ≦0.5 in the Gonnet PAM 250 matrix; and a space identifies a non-conserved amino acid variation. Based on the above alignment, the human and mouse sequences share PR domains that are about 95% identical.

Based on the foregoing alignment, it is contemplated that the present invention can be practiced with ME proteins that share at least 60% identity, more preferably at least about 70% identity, most preferably at least about 80%, 85%, 90%, or 95% identity with the human or mouse PR domains, where residues critical to PR domain function, such as Y₁₁₁, R₁₇₆, and W₁₄₉ in the mouse ME and Y₁₀₉, R₁₇₄, and W₁₄₇ in the human ME, remain unaltered. It is further contemplated that the present invention can be practiced with ME proteins that share the consensus sequence below (SEQ ID NO: 5):

LRESXMPGAG LGIWTKRKIE XGEKFGPYXG EQRSXLKDXS
YGWEILDEFX NVKFCIDASQ PDVGSWLKYI RFAGCYDQHN
LVACQINDQI FYRVVADIAP GEELLLFMKS

where X can be any amino acid.

The SET domain is present in a variety of nuclear regulatory proteins and in many of these has been shown to have histone methyltransferase (HMT) activity, which is the ability to transfer a methyl group from S-adenosyl methionine (SAM) to lysine residues of specific target proteins. The most commonly investigated and tested target protein for this enzymatic activity is histone, though other proteins may also be targets (e.g., p53; Chuikov et al., “Regulation of p53 Activity Through Lysine Methylation,” Nature 432:353-360 (2004), which is hereby incorporated by reference in its entirety). The potential of the PR domain of ME to harbor histone methyltransferase activity has been addressed, and while one report suggests that it does not in the absence of any other binding partner (Nanjundan et al., “Amplification of MDS1/EVI1 and EVI1 Located in the 3q26.2 Amplicon, is Associated with Favorable Patient Prognosis in Ovarian Cancer,” Cancer Res. 67:3074-3084 (2007), which is hereby incorporated by reference in its entirety), a more recent report confirms that baculovirus-expressed, recombinant ME does possess histone methyltransferase activity (Pinheiro et al., “Prdm3 and Prdm16 are H3K9me1 Methyltransferases Required for Mammalian Heterochromatin Integrity.” Cell 150: 948-960 (2012), which is hereby incorporated by reference in its entirety). Another report suggests the PR domain of ME may function in oligomerization (Nitta et al., “Oligomerization of Evi-1 Regulated by the PR Domain Contributes to Recruitment of Corepressor CtBP,” Oncogene 24:6165-6173 (2005), which is hereby incorporated by reference in its entirety); however, this is controversial (Senyuk et al., “The distal Zinc Finger Domain of AML1/MDS1/EVI1 is an Oligomerization Domain Involved in Induction of Hematopoietic Differentiation Defects in Primary Cells In Vitro,” Cancer Res. 65:7603-7611 (2005), which is hereby incorporated by reference in its entirety). Instead, the accompanying Examples explain why it is believed that ME does, indeed, possesses methyltransferase activity, either alone or in the presence of a binding partner.

The methods of the present invention utilize an inhibitor of ME protein activity. These inhibitors can interact directly or indirectly with the ME protein to completely or partially inhibit ME protein function within the targeted cancer cells. As demonstrated in the accompanying Examples, ME protein function is critical to the survival of certain types of cancer cells (i.e., ME-dependent cancer cells). Alternatively, the inhibitors can interfere with ME protein expression so as to diminish or abolish the level of ME protein present in the targeted cancer cells.

In accordance with one embodiment, the inhibitor of ME protein activity is an antibody or antibody fragment or antibody mimic that binds specifically to a PR domain of the ME protein. Because of their specificity, monoclonal antibodies are particularly desirable; however, mono-specific polyclonal antibody populations can also be used. Antibodies are preferably directed to the binding of the PR domain at or near residues critical to PR domain function, such as Y₁₁₁, R₁₇₆, and W₁₄₉ in the mouse ME and Y₁₀₉, R₁₇₄, and W₁₄₇ in the human ME. Binding to the PR domain at or near these locations should interfere with ME activity and inhibit its function in the cancer cells.

Methods for monoclonal antibody production may be achieved using the techniques described herein or other well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (i.e., the ME protein or specific peptide fragments thereof, such as the PR domain, alone or fused with a suitable immunogenic conjugate). Briefly, the antigen of interest is administered subcutaneously to New Zealand white rabbits which have first been bled to obtain pre-immune serum. The antigen can be injected at a total volume of 100 μl per site at six different sites, and each injection will contain synthetic surfactant adjuvant pluronic polyols, or pulverized acrylamide gel containing the purified antigen. The rabbits are then bled two weeks after the first injection and periodically boosted with the same antigen three times every six weeks. A sample of serum is then collected 10 days after each boost. Ultimately, the rabbits are euthanized prior to harvesting lymphocytes from the spleen.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur. J. Immunol. 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

In addition to whole antibodies, the present invention encompasses binding portions of such antibodies. Such binding portions include the monovalent Fab fragments, Fv fragments (e.g., single-chain antibody, scFv), and single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES:PRINCIPLES AND PRACTICE 98-118 (Academic Press, 1983) and Ed Harlow and David Lane, ANTIBODIES: A LABORATORY MANUAL (Cold Spring Harbor Laboratory, 1988); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), all of which are hereby incorporated by reference in their entirety, or other methods known in the art.

It may further be desirable, especially in the case of antibody fragments, to modify the antibody to increase its serum half-life. This can be achieved, for example, by incorporation of a salvage receptor binding epitope into the antibody fragment by mutation of the appropriate region in the antibody fragment or by incorporating the epitope binding site into a peptide tag that is then fused to the antibody fragment at either end or in the middle (e.g., by DNA or peptide synthesis).

Antibody mimics are also suitable for use in accordance with the present invention. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as antibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

In preparing these antibody mimics the CDR sequences of the V_{H} and/or V_{L} chains (from monoclonal antibodies raised again ME or its PR domain) can be grafted into the variable loop regions of these antibody mimics. The grafting can involve a deletion of at least two amino acid residues up to substantially all but one amino acid residue appearing in a particular loop region along with the substitution of the CDR sequence. Insertions can be, for example, an insertion of the CDR domain at one or more locations of a particular loop region. The deletions, insertions, and replacements on the polypeptides can be achieved using recombinant techniques beginning with a known nucleotide sequence.

In accordance with another embodiment, the inhibitor of ME protein activity is an anti-ME nucleic acid aptamer that binds specifically to a PR domain of the ME protein.

Anti-ME nucleic acid aptamers can be formed of DNA or RNA, and are characterized by specificity for the ME PR domain. Aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges.

Nucleic acid aptamers include multivalent aptamers and bivalent aptamers. Methods of making bivalent and multivalent aptamers and their expression in multi-cellular organisms are described in U.S. Pat. No. 6,458,559 to Shi et al., which is hereby incorporated by reference in its entirety. A method for modular design and construction of multivalent nucleic acid aptamers, their expression, and methods of use are described in U.S. Patent Publication No. 2005/0282190 to Shi et al, which is hereby incorporated by reference in its entirety.

Identifying suitable nucleic acid aptamers that inhibit the activity of ME, as described above, basically involves selecting aptamers that bind ME, specifically its PR domain, with sufficiently high affinity (e.g., Kd=20-50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell Biol. 17:1649-1657 (1997); Shi, “Perturbing Protein Function with RNA Aptamers” (thesis, Cornell University) microformed on (University Microfilms, Inc. 1997), each of which is hereby incorporated by reference in their entirety). For example, identifying suitable nucleic acid aptamers can be carried out using an established in vitro selection and amplification scheme known as SELEX using the PR domain of ME as the target for aptamer selection. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk & Gold, “Systematic Evolution of Ligands by Exponential Enrichment: RNA Ligands to Bacteriophage T4 DNA Polymerase,” Science 249:505-510 (1990), each of which is hereby incorporated by reference in its entirety. The SELEX procedure can be modified so that an entire pool of aptamers with binding affinity can be identified by selectively partitioning the pool of aptamers. This procedure is described in U.S. Patent Application Publication No. 2004/0053310 to Shi et al., which is hereby incorporated by reference in its entirety.

Once selected for their binding affinity, aptamers that bind to and inhibit activity of ME can be identified for use in the present invention based on their ability to inhibit cancer cell survival in vitro or in vivo as demonstrated in the accompanying Examples for shRNA.

In accordance with another embodiment, the inhibitor of ME protein activity is a dominant negative ME polypeptide that interferes with ME protein interaction with its putative binding partner. One example of such a ME polypeptide is the PR domain, identified above, which contains the putative binding pocket that interacts with the interacting partner of ME. Any dominant negative polypeptides that inhibit ME activity can also be screened in vitro and in vivo to assess their ability to inhibit cancer cell survival as described in the accompanying Examples for shRNA.

In accordance with a further embodiment, the inhibitor of ME protein activity can be a small molecule ME inhibitor that disrupts ME protein interaction with its putative binding partner. One approach for measuring such disruption is via a transcriptional readout. Any small molecules that inhibit ME activity can also be screened in vitro and in vivo to assess their ability to inhibit cancer cell survival as described in the accompanying Examples for shRNA.

A further embodiment of the inhibitor of ME protein activity is an inhibitory nucleic acid (RNAi) molecule that interferes with ME expression to cause a reduction in ME expression levels and, hence, overall levels of ME activity.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. It will be understood that in this specification the terms siRNA and RNAi are interchangeable. Furthermore, as is well-known in this field RNAi technology may be effected by siRNA, miRNA or shRNA or other RNAi inducing agents. Although siRNA will be referred to in general in the specification. It will be understood that any other RNA inducing agent may be used, including shRNA, miRNA or an RNAi-inducing vector whose presence within a cell results in production of an siRNA or shRNA targeted to a target MECOM or ME-encoding transcript.

RNA interference is a multistep process and is generally activated by double-stranded RNA (dsRNA) that is homologous in sequence to the targeted MECOM transcript. Introduction of long dsRNA into the cancer cells expressing MECOM leads to the sequence-specific degradation of those MECOM gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted ME-encoding RNA molecule. The endonuclease activity hydrolyzes the ME-encoding RNA at the site where the antisense strand is bound. Therefore, RNAi is an antisense mechanism of action, as a single stranded (ssRNA) RNA molecule binds to the target ME-encoding RNA molecule and recruits a ribonuclease that degrades the ME-encoding RNA.

Preferably, siRNA, miRNA or shRNA targeting ME-encoding RNA are used.

Exemplary RNAi for ME (and Evi1) include, without limitation, an shRNA molecule encoded by the sequences:

5′-ATCCGAGCGCCTGACATCCACGAAGAACTTCAAGAGAGTTCTTCGTGG ATGTCAGGCGCtttttACGCGTG-3′ (SEQ ID NO: 6), corresponding to by 511-532 of GenBank sequence M21829, and designated as sh11, which has the following structure:

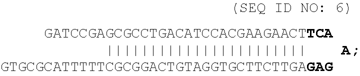

and
5′-GATCCGAGTTGTTGGATGAGGAGGATGAATTCAAGAGATTCATCCTC CTCATCCAACAACtttttACGCGTG-3′ (SEQ ID NO: 7), corresponding to by 3139-3167 of M21829 and designated as sh54, which has the following structure:

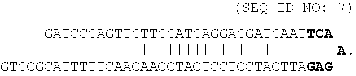

These sequences were selected using software at the RNAi OligoRetriever Database.

Exemplary shRNA specific for the murine ME PR domain include, without limitation:

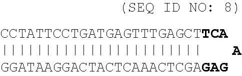

This sequence corresponds to basepairs 229-250 of the composite (CN697723-M21829) sequence listed above, and was obtained from GeneLink, Inc.

Exemplary RNAi specific for the ME PR domain region include, without limitation: RNAi targeting nt 494-514 of the above identified human ME-encoding sequence (target=GCCAGATAAATGATCAGATAT) (SEQ ID NO: 9), which was identified using the Invitrogen RNAi Designer website; RNAi targeting nt 403-421 of the above-identified human ME-encoding sequence (uuCUGCAUAGAUGCCAGUCAAcc) (SEQ ID NO: 10), which was identified by the Kribbs siRNA design website; and RNAi targeting nt 266-284 of the above-identified human ME-encoding sequence (ggGCAGGACUAGGAAUAUGGAcc) (SEQ ID NO: 11), which was identified by the Kribbs siRNA design website.

In certain embodiments, delivery of the inhibitor of ME protein activity can be achieved systemically using a non-targeted delivery system. Non-targeted delivery of RNAi specific for the ME PR domain region is feasible, because blocking ME function in normal cells is not detrimental. This is confirmed via MDS1 KO homozygous mice, which are perfectly viable. For example, delivery agents for the inhibitor of ME protein activity may include those selected from the following non-limiting group of cationic polymers, modified cationic polymers, peptide molecular transporters, lipids, liposomes and/or non-cationic polymers. Viral vector delivery systems may also be used for RNAi inducing agents. For example, an alternative delivery route includes the direct delivery of RNAi inducing agents (including siRNA, shRNA and miRNA) and even anti-sense RNA (asRNA) in gene constructs followed by the transformation of cells within bone marrow compartment with the resulting recombinant DNA molecules. This results in the transcription of the gene constructs encoding the RNAi inducing agent, such as siRNA, shRNA and miRNA, or even asRNA and provides for the transient and stable expression of the RNAi inducing agent in those transformed cells of the bone marrow compartment.

In other embodiments, a targeted delivery system or complex can be used to deliver the inhibitor of ME protein activity primarily or exclusively to the targeted cancerous cells. There are a number of targeted delivery vehicles that use peptide/receptor-, antibody-, or aptamer-mediated delivery of molecular complexes or particles to the cancer cells of interest.

One example of a peptide/receptor targeting system is described by Jin et al. (“Targeted Delivery of Antisense Oligodeoxynucleotide by Transferrin Conjugated pH-sensitive Lipopolyplex Nanoparticles: A Novel Oligonucleotide-based Therapeutic Strategy in Acute Myeloid Leukemia,” Mol. Pharm. 7(1):196-206 (2010), which is hereby incorporated by reference in its entirety). Jin et al. describes the effective use of transferrin conjugated pH-sensitive lipopolyplex nanoparticles (LPs) that incorporate a therapeutic nucleic acid molecule and can release the same at acidic endosomal pH to facilitate the cytoplasmic delivery of the therapeutic nucleic acid molecule after endocytosis.

Several examples describe the use of monoclonal anti-CD-33 antibody-mediated delivery of therapeutic complexes to AML cells. One example, reported by Simard et al. (“In vivo Evaluation of pH-sensitive Polymer-based Immunoliposomes Targeting the CD33 Antigen,” Mol. Pharm. 7(4):1098-1107 (2010), which is hereby incorporated by reference in its entirety), involves pH-sensitive immunoliposomes obtained by anchoring a copolymer of dioctadecyl, N-isopropylacrylamide and methacrylic acid in bilayers of PEGylated liposomes and coupling the whole anti-CD33 monoclonal antibody (mAb) or its Fab′ fragments, which was then used to deliver a payload of 1-beta-d-arabinofuranosylcytosine (ara-C) to human myeloid leukemia cells in a mouse model. Another example, reported by Rothdiener et al., (“Targeted Delivery of SiRNA to CD33-positive Tumor Cells with Liposomal Carrier Systems,” J. Control Release 144(2):251-258 (2010), which is hereby incorporated by reference in its entirety), involves siRNA-loaded immunoliposomes (IL) and immunolipoplexes (ILP) containing free or polyethylene imine (PEI)-complexed into PEGylated liposomes endowed with an anti-CD33 single-chain Fv fragment (scFv). One well known anti-CD33 mAb is gemtuzumab.

Several examples describe the use of monoclonal anti-CD-19 antibody-mediated delivery of therapeutic complexes to ALL cells. One example, reported by Harata et al., (“CD19-targeting Liposomes Containing Imatinib Efficiently Kill Philadelphia Chromosome-positive Acute Lymphoblastic Leukemia Cells,” Blood 104(5):1442-1449 (2004), which is hereby incorporated by reference in its entirety), involves immunoliposome carrying anti-CD19 antibody (CD19-liposomes) for the delivery of the BCR-ABL tyrosine kinase inhibitor Imatinib to Philadelphia chromosome-positive acute lymphoblastic leukemia at near 100% internalization efficiency.

As an alternative to antibody-based targeting of the ME-expressing cancer cells, aptamers can also be used. One exemplary aptamer that targets myeloid leukemic cells is KH1C12 [5′-dAdTdCdCdAdGdAdGdTdGdAdCdGdCdAdGdCdAdTdGdCdCdC dTdAdGdTdTdAdCdTdAdCdTdAdCdTdCdTdTdTdTdTdAdGdCdAdAdAdCdGdCd CdCdTdCdGdCdTdTdTdGdGdAdCdAdCdGdGdTdGdGdCdTdTdAdGdT-3′] (SEQ ID NO: 12). This aptamer can be joined to an aptamer or RNAi molecule that inhibits ME expression or activity, as described. Before joining two functional RNA molecules, it is often beneficial to first predict the secondary structures of the chimeric nucleic acid molecule to ensure that their combination is unlikely to disrupt their secondary structures. Secondary structure predictions can be performed using a variety of software including, without limitation, RNA Structure Program (Dr. David Mathews, University of Rochester) and MFold (Dr. Michael Zuker, The RNA Institute, SUNY at Albany), among others. If the secondary structure predictions suggest no problems, then the chimeric nucleic acid molecules can be generated. Double-stranded DNA templates can be prepared by cloning their PCR products into a cloning vector and using the clones as templates for PCR with the appropriate primers (e.g., 5′ primer for one aptamer portion and 3′ primer for the other aptamer portion). These same primers can be used to generate the chimeric DNA template for transcription, and in vitro transcription can be carried out using standard procedures to obtain the RNA chimeras, which can then be gel purified prior to use.

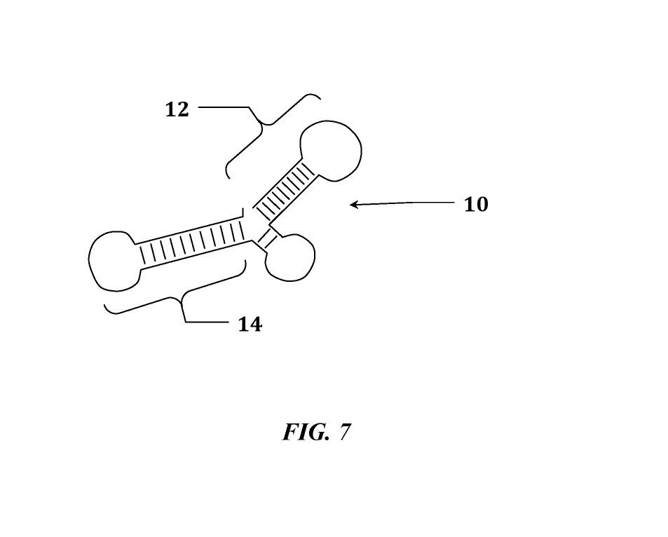

One embodiment of the chimeric therapeutic agent (10) shown in FIG. 7 includes the AML-specific aptamer KH1C12 (12) and either an ME-inhibiting aptamer or ME-inhibiting RNAi molecule, generally denoted at (14), which is targeted to AML cells recognized by the KH1C12 aptamer. Upon binding of the KH1C12 aptamer to the AML cell, the cancer cell will take up the molecule and the ME-inhibiting component (14) will interfere with ME expression or activity. As demonstrated in the accompanying Examples, disruption of ME expression or activity will diminish both proliferation and survival of the targeted cancer cells.

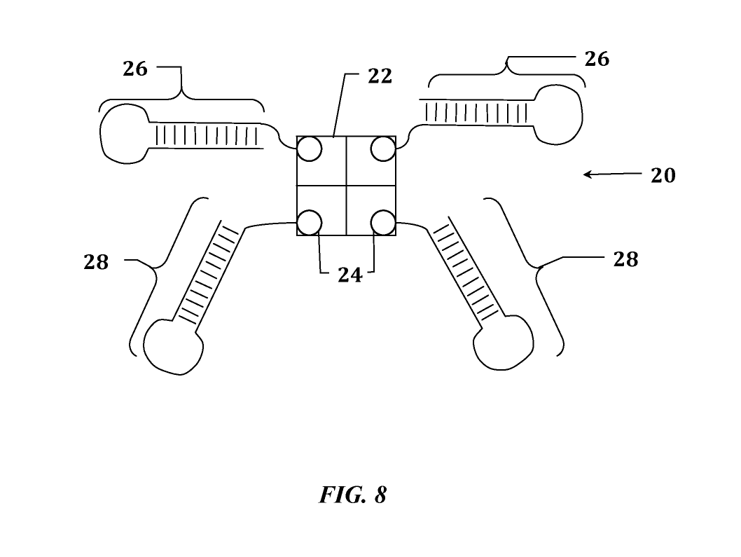

In another approach for targeted delivery of the therapeutic agent, illustrated in FIG. 8, a conjugated aptamer molecule (20) is provided. In this embodiment, one or more AML-specific KH1C12 aptamers (26) and one or more ME-inhibiting aptamer or ME-inhibiting RNAi molecule, generally denoted at (28), form the functional components of the conjugate (20). (In FIG. 8, two of each are shown.) All four of these molecules are biotinylated (24), and the conjugate is formed upon incubation of the biotinylated aptamers/RNAi molecules with streptavidin (22). Biotinylation of the aptamers at their 3′ ends is known not to interfere with the activity of these RNA molecules (see Chu et al., “Aptamer Mediated siRNA Delivery,” Nucl. Acids Res. 34(10):e73 (2006), which is hereby incorporated by reference in its entirety). Conjugate (20) is targeted to AML cells recognized by the KH1C12 aptamer. Upon binding of the KH1C12 aptamer to the AML cell, the cancer cell will take up the molecule and the ME-inhibiting component (28) will interfere with ME expression or activity. As demonstrated in the accompanying Examples, disruption of ME expression or activity will diminish both proliferation and survival of the targeted cancer cells.

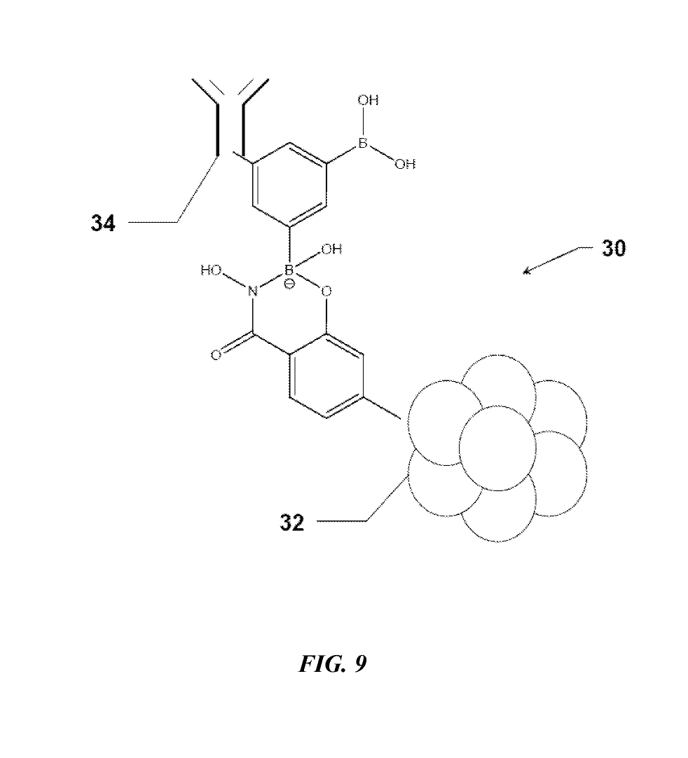

In a further approach for targeted delivery of the therapeutic agent, illustrated in FIG. 9, a conjugate (30) includes a polycation-aptamer or RNAi vector (32) linked via phenyl(di)boronic acid-salicylhydroxamic acid assembly to an antibody (34) that is specific for a cancer cell surface marker (e.g., CD-33, CD-19, CD-20 as described above). In this embodiment, the phenyl(di)boronic acid is first coupled to the antibody via a PEG linker using the methodology of Moffatt et al., “Successful In Vivo Tumor Targeting of Prostate-specific Membrane Antigen with a Highly Efficient J591/PEI/DNA Molecular Conjugate,” Gene Therapy 13:761-772 (2006), which is hereby incorporated by reference in its entirety. The salicylhydroxamic acid is coupled to polyethyleneimine (PEI), a polycation, using the procedures of Moffatt et al. (“Successful In Vivo Tumor Targeting of Prostate-specific Membrane Antigen with a Highly Efficient J591/PEI/DNA Molecular Conjugate,” Gene Therapy 13:761-772 (2006), which is hereby incorporated by reference in its entirety), and thereafter the aptamer/RNAi molecule can be introduced to the SHA-PEI solution to form the self-assembled conjugate (30).

One embodiment of this type of therapeutic agent includes a CD-33-specific monoclonal antibody and one or more ME-specific RNAi molecules in the PEI matrix, which are conjugated together via PDB-SHA bridge. This conjugate is targeted to CD-33-positive AML cells. Upon binding of the antibody to the CD-33-positive AML cells, the cancer cell will take up the conjugate and the ME-specific RNAi will interfere with ME expression and activity. As demonstrated in the accompanying Examples, disruption of ME expression or activity will diminish both proliferation and survival of the targeted cancer cells.

Another embodiment of this type of therapeutic agent includes a CD-19-specific monoclonal antibody and one or more ME-specific RNAi molecules in the PEI matrix, which are conjugated together via PDB-SHA bridge. This conjugate is targeted to CD-19-positive AML cells. Upon binding of the antibody to the CD-19-positive AML cells, the cancer cell will take up the conjugate and the ME-specific RNAi will interfere with ME expression and activity. As demonstrated in the accompanying Examples, disruption of ME expression or activity will diminish both proliferation and survival of the targeted cancer cells.

Another embodiment of this type of therapeutic agent includes a CD-20-specific monoclonal antibody and one or more ME-specific RNAi molecules in the PEI matrix, which are conjugated together via PDB-SHA bridge. This conjugate is targeted to CD-20-positive AML cells. Upon binding of the antibody to the CD-20-positive AML cells, the cancer cell will take up the conjugate and the ME-specific RNAi will interfere with ME expression and activity. As demonstrated in the accompanying Examples, disruption of ME expression or activity will diminish both proliferation and survival of the targeted cancer cells.

Polymeric nanoparticles can be targeted to cell-surface marked using aptamers designed using the SELEX procedure (Farokhzad et al., “Targeted Nanoparticle-aptamer Bioconjugates for Cancer Chemotherapy in vivo,” Proc. Natl. Acad. Sci. USA 103(16):6315-6320 (2006), which is hereby incorporated by reference in its entirety). Nanoparticles and microparticles may comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath et al., “Biodegradable Polymeric Nanoparticles as Drug Delivery Devices,” J. Control Release 70(1-2):1-20 (2001), which is hereby incorporated by reference in its entirety. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen et al., “Colloids and Surfaces,” Biointerfaces 16(1-4):3-27 (1999), which is hereby incorporated by reference in its entirety. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(D,L-lactide) may be used while the lipid monolayer is typically composed of a mixture of lipids. Suitable methods of preparation are similar to those referenced above for polymer nanoparticles. Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese et al, “Bioconjugation in Pharmaceutical Chemistry,” IL Farmaco 54(8):497-516 (1999), which is hereby incorporated by reference in its entirety.

The various therapeutic agents of the present invention can be administered systemically (e.g., orally, parenterally, subcutaneously, intravenously, intramuscularly, intraperitoneally, by intranasal instillation, by implantation, by intracavitary or intravesical instillation, intraocularly, intraarterially, intralesionally, transdermally, by application to mucous membranes, or by introduction into one or more lymph nodes), and/or directly into a bone marrow compartment.

For the use in accordance with the invention, the appropriate dosage of the therapeutic agent, e.g. inhibitor of ME activity, will, of course, vary depending upon, for example, the particular agent to be employed, the host, the mode of administration and the severity of the cancerous condition being treated, and the effects desired. Satisfactory results can be obtained at dosages from about 0.1 mg to about 1000 mg, preferably from 1 to 100 mg, more preferably 20-50 mg. For example dosages can be 0.3 mg/kg body weight, 1 mg/kg body weight, 3 mg/kg body weight, 5 mg/kg body weight or 10 mg/kg body weight, or within the range of 1-10 mg/kg body weight. Administration may be in a single dose or in several doses over a period of time as long as may be indicated in relation to the time the disease is clinically evident or prophylactically to suppress further clinical relapse, for example a dose from about 5 up to about 100 mg, may be administered once a month, until control or amelioration of the disease is achieved. A preferred dosage regimen comprises administration of 20-50 mg of the therapeutic inhibitor of ME activity every two weeks or once a month. An exemplary treatment regime entails administration once per week, once every two weeks, once every three weeks, once every four weeks, once a month, once every 3 months or once every three to 6 months.

For oral therapeutic administration (e.g., sublingual delivery), the therapeutic agents may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The therapeutic agent may also be administered parenterally. Solutions or suspensions of these therapeutic agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol), suitable mixtures thereof, and vegetable oils.

The therapeutic agents of the present invention may also be administered directly to the airways in the form of an aerosol or via a lung surfactant formulation. For use as aerosols, the inhibitors of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer. A number of commercially available lung surfactant formulations exist, including synthetic surfactant formulations and exogenous formulations.

Persons of skill in the art are readily able to test and assess optimal dosage schedules based on the balance of efficacy and any undesirable side effects. The optimal dosage of each type of ME inhibitor will vary, of course, and the minimal effective dose will be administered for therapeutic regimen.

Sustained release formulations include implantable devices that include a slow-dissolving polymeric matrix and the therapeutic agent of the invention retained within the polymeric matrix. The matrix can be designed to deliver substantially the entire payload of the vehicle over a predetermined period of time, such as about one to two weeks or about one to three months.

As indicated above, the therapeutic agents of the present invention can be administered in using a delivery vehicle for passive or targeted delivery to cancer cells that express the ME protein for its continued survival. As indicated, any suitable passive or targeted delivery vehicle can be employed, including liposomes, polymeric nanoparticles, chimeric proteins, polyethylene glycol conjugates, and oligoarginine.

The therapeutic agents of the present invention can also be used alone or in combination with one or more additional therapies, including without limitation, bone marrow transplant, chemotherapies, radiation therapies, immunotherapies, and combinations thereof with or without adjuvants that enhance the efficacy of those therapies, such as CXCR4 antagonists of the type described in O'Callaghan et al., “Targeting CXCR4 with Cell-penetrating Pepducins in Lymphoma and Lymphocytic Leukemia,” Blood 119(7):1717-1725 (2012); Parameswaran et al., “Treatment of Acute Lymphoblastic Leukemia with an rGel/BLyS Fusion Toxin,” Leukemia doi: 10.1038/leu.2012.54 (February 2012), each of which is hereby incorporated by reference in its entirety.

Exemplary conventional and experimental therapies for AML include, without limitation, cytarabine and anthracycline chemotherapy regimen, gemtuzumab ozogamicin, stem cell transplant, clofarabine, farnesyl transferase inhibitors, decitabine, MDR1 inhibitors, arsenic trioxide, all-trans retinoic acid.

Exemplary conventional and experimental therapies for ALL include, without limitation, one or more of the following: radiation therapy on affected bone areas; combinations of prednisone or dexamethasone, vincristine, asparaginase, and daunorubicin to induce remission; combinations of vincristine, cyclophosphamide, cytarabine, etoposide, thioguanine, or mercaptopurine during intensification, or for CNS protection intrathecal methotrexate or cytarabine, alone or combined, and either with or without cranio-spinal irradiation; intrathecal administration of hydrocortisone, methotrexate, and cytarabine for CNS relapse; and the scheduled separate administration of mercaptopurine, methotrexate, vincristine and corticosteroids for maintenance therapy.

### EXAMPLES

The Examples set forth below are for illustrative purposes only and are not intended to limit, in any way, the scope of the present invention.

### Materials and Methods for Examples 1-5

Mice—

ME^{m1} and ME^{fl4} (Evi1^{fl3} previously) alleles (Zhang et al., “PR Domain—Containing Mds1-Evi1 is Critical for Long-Term Hematopoietic Stem Cell Function,” Blood 118(14):3853-3861 (2011), which is hereby incorporated by reference in its entirety) and Esr-Cre (Hayashi et al., “Efficient Recombination in Diverse Tissues by a Tamoxifen-Inducible Form of Cre: A Tool for Temporally Regulated Gene Activation/Inactivation in the Mouse,” Dev. Biol. 244:305-318 (2002), which is hereby incorporated by reference in its entirety) have been described. All procedures were approved by an animal care and use committee.

Retroviral Constructs—

The MLL-AF9 expression construct (Barabé et al., “Modeling the Initiation and Progression of Human Acute Leukemia in Mice,” Science 316:600-604 (2007), which is hereby incorporated by reference in its entirety) was moved to mCherry. pMIGR1-ME (p1183) was made by inserting MDS1 (Zhang et al., “PR Domain—Containing Mds1-Evi1 is Critical for Long-Term Hematopoietic Stem Cell Function Blood. 118(14):3853-3861 (2011), which is hereby incorporated by reference in its entirety) into BglII-cut p854 (Zhang et al., “Targeting a DNA Binding Motif of the EVI1 Protein by a Pyrrole-Imidazole Polyamide,” Biochemistry 50(48):10431-10441 (2011), which is hereby incorporated by reference in its entirety) pMIGR1-MDS (p1211) was made by PCR (5′atgaattcgcatgagatccaaaggcagggc3′/5′ atgaattatcacctggtctcccatccatagctg3′) (SEQ ID NOS: 13 and 14, respectively) and cloning.

Serial Replating Assay—

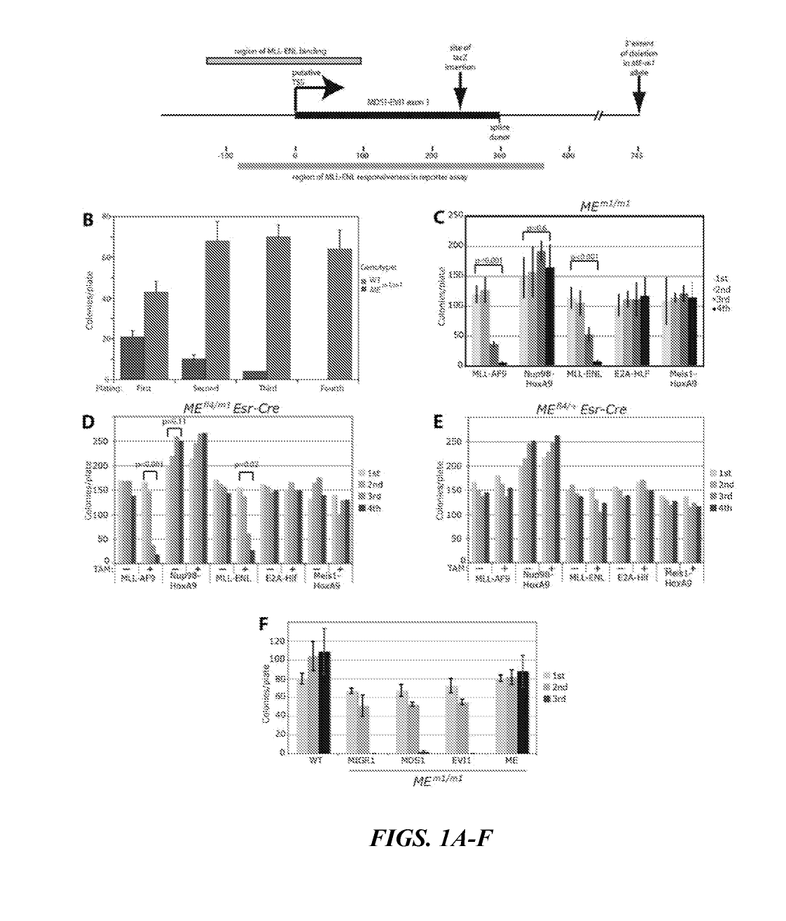

Lineage-negative/Sca-1⁺/c-kit⁺ (LSK) cells were isolated (t=0 hr), infected (Zhang et al., “PR Domain—Containing Mds1-Evi1 is Critical for Long-Term Hematopoietic Stem Cell Function,” Blood 118(14):3853-3861 (2011), which is hereby incorporated by reference in its entirety) with retrovirus (t=15 hr), sorted (t=63 hr), and plated in M3434 (StemCell). For FIG. 1F, add-back infections were performed following MLL-AF9 sort (t=64 h), sorted for GFP (t=90 h) and plated in M3434 (Lavau et al, “Immortalization and Leukemic Transformation of a Myelomonocytic Precursor by Retrovirally Transduced HRX-ENL,” EMBO J. 16:4226-4237 (1997), which is hereby incorporated by reference in its entirety).

In Vivo Leukemogenesis Assay—

Primary leukemias were harvested, explanted to culture, +/−4-OH TAM; 10⁶ spleen cells/mouse were transplanted into sublethally irradiated secondary recipient mice via tail vein injection. Leukemia development in transplanted mice was monitored by assessing total blood counts (CBC) every week.

### Example 1

### Knock-in of lacZ into Mds1 Exon 1

To assess the role of the Mds1 gene in hematopoiesis, a targeted disruption of the Mds1 was created by inserting a promoterless lacZ gene into the first coding exon in the mouse designated Mds1^{tm1ap} (herein termed ME^{m1}; Barabé et al., “Modeling the Initiation and Progression of Human Acute Leukemia in Mice,” Science 316:600-604 (2007), which is hereby incorporated by reference in its entirety). The structure of the ME^{m1} allele is such that the expression of lacZ is under the control of the Mds1 promoter, and the splice donor is deleted. The β-galactosidase protein produced contains a nuclear localization signal from SV40 virus; staining for the enzyme was found to be concentrated to the nucleus. Matings between heterozygous mice generated live born homozygous ME^{m1/m1} mice close to the expected Mendelian frequency (Barabé et al., “Modeling the Initiation and Progression of Human Acute Leukemia in Mice,” Science 316:600-604 (2007), which is hereby incorporated by reference in its entirety). ME^{m1/m1} mice appeared normal at birth, but grew more slowly than wildtype and all developed a progressive kyphosis (Zhang et al., “Targeting a DNA Binding Motif of the EVI1 Protein by a Pyrrole-Imidazole Polyamide,” Biochemistry 50(48):10431-10441 (2011), which is hereby incorporated by reference in its entirety). To assess allelism with Evi1, matings of Mds1^{m1/+} and Evi1^{+/-} mice (Lavau et al, “Immortalization and Leukemic Transformation of a Myelomonocytic Precursor by Retrovirally Transduced HRX-ENL,” EMBO J. 16:4226-4237 (1997), which is hereby incorporated by reference in its entirety) were performed which revealed that no mice carrying both mutations were identified (p<0.0001). These data indicate an essential role for the combination of both ME and Evi1 transcripts during development.

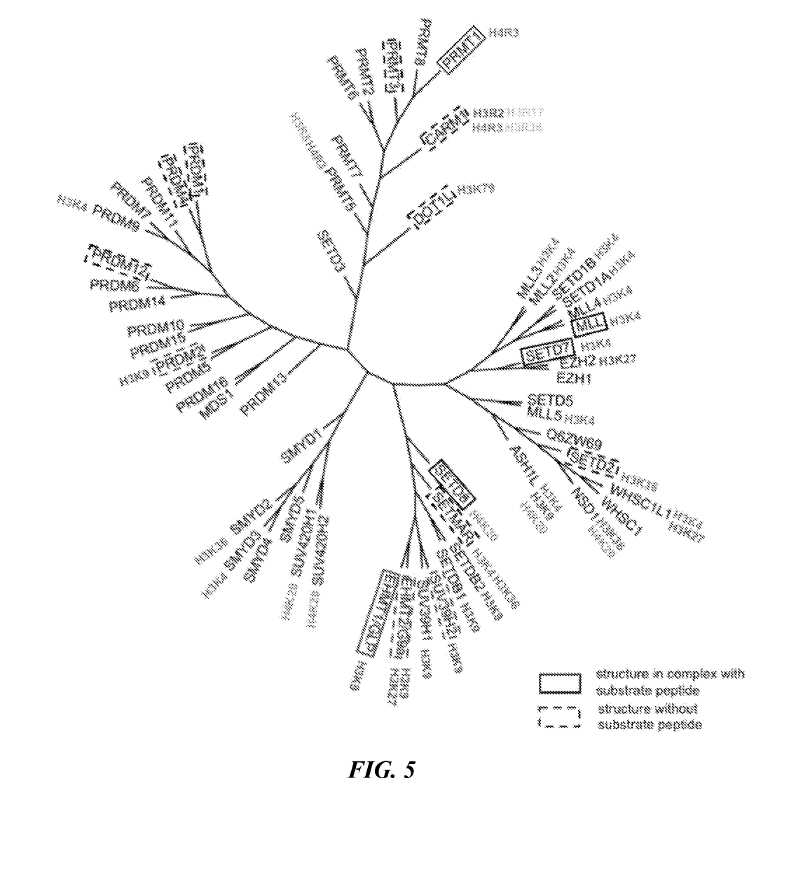

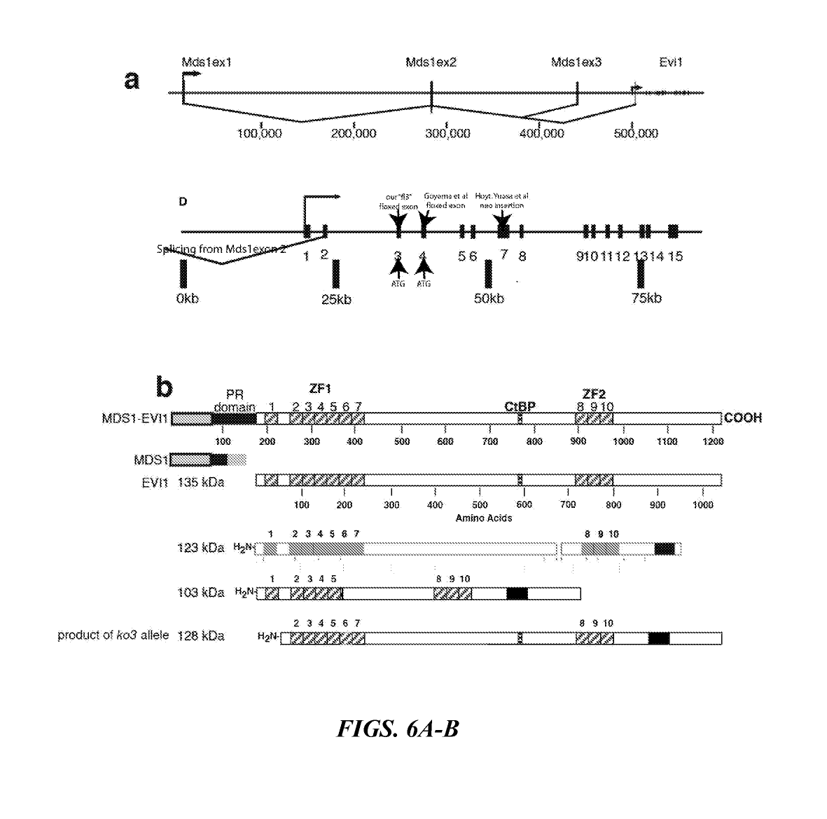

It is known that Evi1 is one of the highest upregulated genes in hematopoietic stem and progenitor cells of preleukemic MLL-AF9 knockin mice (Silverman et al., “Intensified Therapy for Infants With Acute Lymphoblastic Leukemia: Results From the Dana-Farber Cancer Institute Consortium,” Cancer 80(12):2285-2295 (1997), which is hereby incorporated by reference in its entirety); however this study did not distinguish Evi1 from ME mRNA transcripts. While conditional knockout studies deleting exon 4 of Evi1 (FIG. 5) have shown that MLL-AF9 transformation is dependent on an intact Mecom gene (Lavau et al., “Immortalization and Leukemic Transformation of a Myelomonocytic Precursor by Retrovirally Transduced HRX-ENL,” EMBO J. 16:4226-4237 (1997), which is hereby incorporated by reference in its entirety), this exon is required for both ME and Evi1 transcripts (FIGS. 6A-B); as such, it was not possible to discern which transcript was responsible for the resistance to transformation by MLL-AF9. Herein, ME is identified as being a critical downstream component in MFP-induced transformation. Further, it is shown that within ME, it is the PR domain that is critical. The mutagenesis and in silico structural analysis of this domain strongly suggests that its function in MFP-induced transformation depends on a substrate binding pocket, and thus, is likely druggable.

### Example 2

### Knockout of Mds1 Isoforms Abrogates the Ability of MLL-AF9 to Transform Bone Marrow Progenitors; Add-Back Assay Shows Primary Role for PR Domain of ME Isoform

To test the role of ME in MLL-AF9 leukemogenesis, bone marrow cells from ME^{m1/m1} mice were infected which bear a lacZ insertion in exon 1 of Mds1 (FIG. 1A) and lack MDS1 and ME, but express normal levels of EVI1 and serial replating assay was performed (Lavau et al, “Immortalization and Leukemic Transformation of a Myelomonocytic Precursor by Retrovirally Transduced HRX-ENL,” EMBO J. 16:4226-4237 (1997); Dobson et al., “The mll-AF9 Gene Fusion in Mice Controls Myeloproliferation and Specifies Acute Myeloid Leukaemogenesis,” Embo J. 18(13):3564-3574 (1999); Krivtsov et al., “Transformation From Committed Progenitor to Leukaemia Stem Cell Initiated by MLL-AF9,” Nature 442(7104):818-822 (2006); and Somervaille et al., “Identification and Characterization of Leukemia Stem Cells in Murine MLL-AF9 Acute Myeloid Leukemia,” Cancer Cell 10:257-268 (2006), which are hereby incorporated by reference in their entirety). While MLL-AF9 induced wildtype cells to form colonies at each cycle, it was unable to transform ME^{m1/m1} cells (FIG. 1B). These findings indicate a dependency of MLL-AF9 transformation on functional Mds1 and/or ME.

Whether the transformation block was specific for MLL-AF9 was then tested. ME^{m1/m1} cells were transduced with MLL-AF9, Nup98-HoxA9, MLL-ENL, E2A-Hlf, or Meis1-HoxA9, and were assayed for transformation. This revealed that cells lacking ME were resistant to transformation only by MLL-AF9 or MLL-ENL (FIG. 1C). Thus, the block to transformation is oncogene-specific. To further test this, ME^{fl4/m1}/Esr-Cre (FIG. 1D) and ME^{fl4/+}/Esr-Cre (FIG. 1E) LSK cells were transduced with the same oncogenes, split into two treatment groups (vehicle or 4-OH-TAM) and assayed for transformation. All vehicle-treated cell samples, regardless of genotype, were fully capable of being transformed with all oncogenes (FIGS. 1D-E), while 4-OH-TAM-treated ME^{fl4/m1}/Esr-Cre cells displayed the same selective resistance to transformation; 4-OH TAM-treated ME^{fl4/+}/Esr-Cre cells were susceptible to transformation by all oncogenes. Together, these data indicate a selective requirement for a functional ME allele for transformation by MFP oncogenes.

To test which isoform of Mecom is required for MLL-AF9 transformation, LSK cells were retrovirally transduced from ME^{m1/m1} mice with MIGR1, or with constructs for MDS1, EVI1 or ME, and then infected with MLL-AF9 retrovirus; transduced cells were assayed for transformation. While neither MDS1 nor EVI1 were able to rescue the deficiency, ME was so able (FIG. 1F), confirming that in the context of the ME^{m1/m1} genotype, ME is the essential isoform that is lacking. Since the major difference between ME and EVI1 is the PR domain, it is clear that this domain is critical for ME activity in the setting of MLL-AF9-induced leukemogenesis.

### Example 3

### Knockout of ME Results in Suppression of MLL-AF9-Leukemia Development in Transplanted Mice

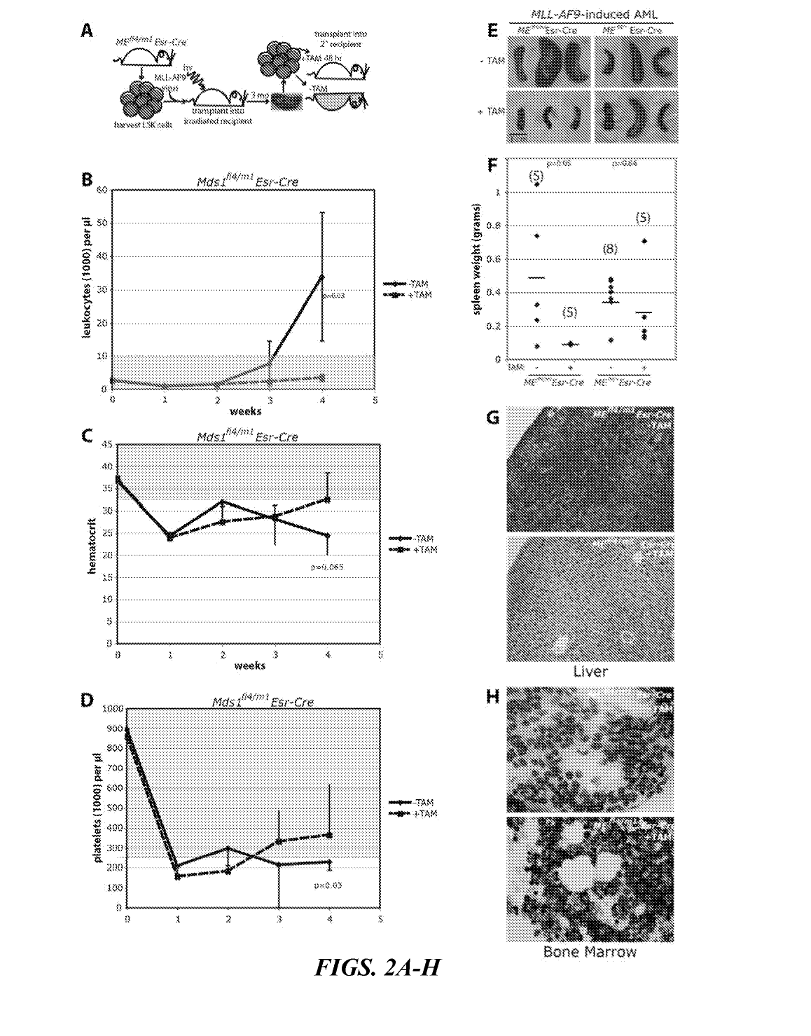

To test if ME is required for transformed MLL-AF9 AML cells to survive in vivo, transplantable leukemias were established and then the gene was deleted and assayed for leukemogenesis by transplantation. ME^{fl4/m1}/Esr-Cre or ME^{fl4/+}/Esr-Cre LSK cells were transduced with MLL-AF9 and transplanted into irradiated recipients. After 3 months, primary AMLs developed, and leukemic spleen cells were harvested, induced with 4-OH-TAM to delete ME (yielding genotypes ME^{ko4/m1} and ME^{ko4/+}, respectively), and injected into irradiated recipients. Weekly CBCs noted an increasing leukocyte count and persistently low hematocrit and platelet count in mice receiving ME^{fl4/m1}/Esr-Cre cells not treated with 4-OH-TAM (FIGS. 2A-C), indicating infiltration of leukemic cells into the blood and bone marrow; in mice receiving 4-OH-TAM-treated ME^{fl4/m1}/Esr-Cre cells, a normal leukocyte level was maintained, and platelets and hematocrit recovered to normal levels. The control group mice receiving ME^{fl4/m1}/Esr-Cre cells with and without 4-OH-TAM treatment all develop leukemia as expected.

Four weeks post-transplant, mice receiving ME^{fl4/+}/Esr-Cre cells with and without 4-OH-TAM treatment, and mice receiving ME^{fl4/m1}/Esr-Cre cells untreated with 4-OH-TAM became moribund; in contrast, the cohort receiving ME^{fl4/m1}/Esr-Cre cells treated with 4-OH-TAM remained healthy. Necropsy confirmed that the moribund mice had widely disseminated disease, with enlarged spleens (FIGS. 2D-E), and, on histopathology, leukemic cell infiltration of bone marrow (FIG. 2F), liver (FIG. 2H) and spleen. In contrast, mice receiving 4-OH-TAM-treated ME^{fl4/m1}/Esr-Cre leukemic cells remained healthy and had normal-sized spleens at necropsy (FIGS. 2C-D), with no or minimal AML infiltration into organs, as assessed by histopathology (FIGS. 2E-H).

Bindels et al. showed overexpression of EVI1 in a subset of MLL fusion protein leukemias; those expressing EVI1 were phenotypically distinct in that they rarely showed monoblastic phenotype (Bindels et al., “EVI1 is Critical for the Pathogenesis of a Subset of MLL-AF9-rearranged AMLs,” Blood 119(24):5838-5849 (2012), which is hereby incorporated by reference in its entirety). Thus, it appears that not all cases of MFP-induced leukemias express the MECOM locus; and that there is a distinct phenotype (monoblastic) when MECOM is not activated. It is likely that MECOM non-expressing MFP-induced leukemias arise via transformation of a cell that is beyond the HSC/CMP stage, at which the MECOM locus is normally silenced (Krivtsov et al., “Cell of Origin Determines Clinically Relevant Subtypes of MLL-Rearranged AML,” Leukemia 27:1-9 (2013), which is hereby incorporated by reference in its entirety). Arai et al. established that MFPs can upregulate transcription of both EVI1 and ME (Arai et al., “Evi-1 is a Transcriptional Target of Mixed-Lineage Leukemia Oncoproteins in Hematopoietic Stem Cells,” Blood 117(23):6304-6314 (2011), which is hereby incorporated by reference in its entirety). While these results reveal an essential role for ME, they do not preclude that EVI1 isoforms are also essential. Nor do they exclude the possibility that it is the ratio of ME to EVI1 that is critical.

Currently there are no clinically available targeted therapies for MLL fusion protein leukemias. Published reports identify alternative targeted therapies for these leukemias: inhibitors of glycerol synthase kinase (GSK) (Wang et al., “Glycogen Synthase Kinase 3 in MLL Leukemia Maintenance and Targeted Therapy,” Nature 455:1205-1209 (2008), which is hereby incorporated by reference in its entirety), DOT1L (Daigle et al., “Selective Killing of Mixed Lineage Leukemia Cells by a Potent Small-Molecule DOT1L Inhibitor,” Cancer Cell 20(1):53-65 (2011), which is hereby incorporated by reference in its entirety), and the MFP-MENIN interaction (Grembecka et al., “Menin-MLL Inhibitors Reverse Oncogenic Activity of MLL Fusion Proteins in Leukemia,” Nat. Chem. Biol. 8(3):277-284 (2012), which is hereby incorporated by reference in its entirety). However, none of these has yet made it to the clinic, and furthermore, each is likely toxic, based on interpolation from genetic studies (Jones et al., “The Histone H3K79 Methyltransferase Dot1L is Essential for Mammalian Development and Heterochromatin Structure,” PLoS Genet. 4(9):e1000190 (2008); Feng et al., “Early Mammalian Erythropoiesis Requires the Dot1L Methyltransferase,” Blood 116(22):4483-4491 (2010); and Chen et al., “The Tumor Suppressor Menin Regulates Hematopoiesis and Myeloid Transformation by Influencing Hox Gene Expression,” Proc. Natl. Acad. Sci. USA 103:1018-1023 (2006), all of which are hereby incorporated by reference in their entirety). Thus, despite these reports, there is still a need for additional avenues for therapeutic intervention. The present invention reveals a novel discovery that ME is necessary for MFP transformation.

The fact that ME but not EVI1 can rescue the mutant phenotype centers attention on the PR domain as being responsible for ME function in the setting of MFP leukemogenesis. Recent studies have shown that this domain harbors H3K9 monomethyltransferase activity, which was expected based on the finding of HMT activity in closely related proteins (Wu et al., “Structural Biology of Human H3K9 Methyltransferases,” PLoS One 1(1):e8570 (2010), which is hereby incorporated by reference in its entirety). This may open an avenue for novel therapeutic intervention in the treatment of MFP leukemias. The fact that ME is a non-essential gene for cellular and organismal survival (Zhang et al., “PR Domain—Containing Mds1-Evi1 is Critical for Long-Term Hematopoietic Stem Cell Function,” Blood 118(14):3853-3861 (2011), which is hereby incorporated by reference in its entirety) provides the expectation that therapies that inhibit its function are likely to be nontoxic and well-tolerated.

### Example 4

### In Silico Analysis of the Structure of the “PR” Domain of MDS1-EVI1

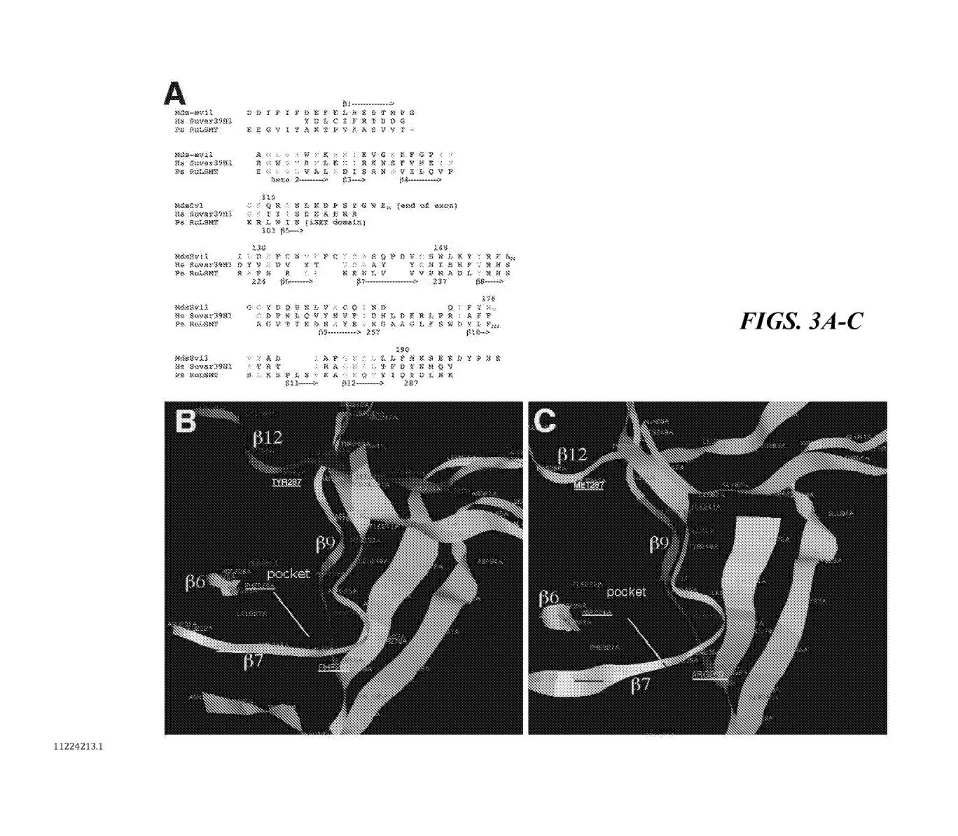

MDS1-EVI1 has a SET-like PR domain at the N-terminus Amino acid alignment (FIG. 3A) shows considerable identity and similarity with Suvar39H1 and RuLSMT, two HMTs with known enzymatic activity (Trievel et al., “Structure and Catalytic Mechanism of a SET Domain Protein Methyltransferase,” Cell 111:91-103 (2002), which is hereby incorporated by reference in its entirety). Previous studies (Nanjundan et al., “Amplification of MDS1/EVI1 and EVI1 Located in the 3q26.2 Amplicon, is Associated With Favorable Patient Prognosis in Ovarian Cancer,” Cancer Res. 67:3074-3084 (2007), which is hereby incorporated by reference) failed to detect HMT activity when the PR domain of ME was purified from E. coli or when expressed in COS7 cells and pulled down with an anti-epitope tag. To further address the possibility of this PR domain possessing HMT activity, in silico structural analysis was performed by “threading” the primary amino acid sequence of the PR domain of MDS1-EVI1 onto the coordinates of a crystallized SET protein (ribulose-1,5-bisphosphate carboxylase/oxygenase large subunit methyltransferase (Rubisco-LSMT)) (Trievel et al., “Structure and Catalytic Mechanism of a SET Domain Protein m=Methyltransferase,” Cell 111:91-103 (2002), which is hereby incorporated by reference in its entirety) using ONO software. This revealed a remarkably close fit with some interesting and important differences between the two domains (FIGS. 3B and 3C).

The essential features of Rubisco-LSMT structure are NH2-SET (N-SET) and COOH-SET (C-SET) domains with an unconserved intervening-SET (i-SET) domain in between (FIG. 3A). The N-SET portion contains β sheets 1 through 5; C-SET contains sheets 6-12. A catalytic pocket which binds the target lysine is caged by four of the β sheets (β6, β7, β9, and β12; FIG. 3B). The pocket is stabilized by inter-sheet interactions, particularly stacking of planar phenylalanine residues F₂₂₄ and F₂₆₉ (diagonal line, FIG. 3B), and several hydrophobic interactions involving W₁₀₄ and V₂₃₃ (FIG. 3B). The binding of SAM is dependent on interactions between SAM and the i-SET domain. A key residue for the enzymatic activity is a tyrosine residue (Y₂₈₇) shown at the top of the pocket in FIG. 3B.

This pocket clearly exists in the PR domain of MDS1-EVI1 and is also caged by four β sheets. The stabilizing interactions are different—the stacking F residues are replaced with a pair of + and − charged-amino acids: designated D₂₂₄ (aa 130 in MDS1-EVI1) and R₂₆₉ (aa 176 in ME) in FIG. 3C. The hydrophobic residues W₁₀₄ and V₂₃₃ have been replaced with charged or polar residues: R (aa 116 in mouse ME) and N (aa 143 in mouse ME), respectively. These changes make the pocket more hydrophilic overall, and larger in size. All of these amino acids are conserved between human and mouse ME, and are therefore believed to be highly conserved among mammalian ME.

A second important difference is that the i-SET domain, on which SAM depends for binding, does not exist in ME. This is true in both human and mouse ME. This indicates that the role of the i-SET domain, if such exists for ME, is likely taken up by an interacting protein or another region of the ME protein. Consistent with this, on the face of the protein where i-SET would be, there are two protruding residues, W₁₄₉ and Q₁₁₅, stabilized by a perfect hydrogen bond, which may provide a docking site for an interacting protein. All of these amino acids are conserved between human and mouse ME, and are therefore believed to be highly conserved among mammalian ME.

A third important difference is that the Y₂₈₇ residue thought to be the critical residue for HMT catalytic activity is changed to a methionine residue (M₁₉₁ of ME). This very likely indicates lack of intrinsic HMT activity, even when ME is within its holocomplex.

In sum, the preceding structural and functional analyses demonstrate a highly related 3-dimensional structure with a larger binding pocket, absence of a full complement of interaction sites for SAM, and an altered residue for the enzymatic activity. Based on these properties, it is believed that these alterations result in an HMT enzyme that requires interaction with another protein for enzymatic activity. Importantly, with this putative structure, specific predictions can be made as to what point mutations will result in loss of activity.

### Example 5

### Add-Back Assay Demonstrates Essential Role for Amino Acids at Putative Catalytic Site of MEPRD

Using the add-back assay in which the rescue of the MLL-AF9 transformation deficiency seen in ME^{m1/m1} mice is scored, the importance of several key amino acids were tested within the MEPRD, with the goal of discerning whether the domain in fact has catalytic activity. Within the binding pocket, the conserved tyrosine (Y₁₁₁) was mutated to alanine: this amino acid is thought to be at the catalytic site, and to be important for specifying whether HMTs function as monomethylases or di-/tri-methylases. R₁₇₆ was also mutated, which is predicted to stabilize the substrate lysine binding pocket through a salt bridge with D₁₃₀. In addition, W₁₄₉ was mutated, which is hypothesized to function in allowing interaction with a binding partner protein that takes on the role of the absent i-SET domain. C₁₅₈, was also mutated, which, it is predicted, is on the opposite side of the domain from the catalytic site, and therefore should not be essential for biologic activity.

FIGS. 4A-E reveal that while wildtype ME protein is able to rescue susceptibility to MLL-AF9-induced transformation as expected, the mutation at the active site (Y₁₁₁A) was devoid of activity, indicating the importance of this residue. Mutation at R₁₇₆ also resulted in the loss of activity, indicating that, if it does play a role in maintaining the pocket structure through interaction with D₁₃₀, the pocket plays a critical role in ME biologic function. Thus, even though the presence of M₁₉₀ may indicate MEPRD lacks HMT enzymatic activity, this data is consistent with MEPRD binding a lysine substrate with Y₁₁₁ playing a role in maintaining its orientation within the pocket. The W149 residue, which the present invention postulates serves as a docking site for an interacting protein, also proved to be essential, as the W149A mutant was devoid of activity. Together, this evidence supports the notion that another protein interacts and plays a critical role in activity. C158A functioned as wildtype, as predicted.

In sum, the analysis of the present invention confirms a structure highly related to enzymatically active SET domains with a larger binding pocket, absence of a full complement of interaction sites for SAM, and an altered residue at the catalytic site. It is therefore likely that this PR domain requires interaction with another protein for activity; this is supported by the loss of activity with the W₁₄₉A mutant. This analysis also indicates that the PR domain of ME may have target(s) other than histone, or perhaps binding residues other than unmethylated lysine.

### Prophetic Example 6

### Construction and Use of Aptamer Conjugate for Targeted Delivery to AML Cells in Animal Models

An aptamer conjugate will be prepared using a 3′ biotinylated AML-specific KH1C12 specific RNA aptamer of SEQ ID NO: 13 and either a 3′ biotinylated ME-inhibiting aptamer or a 3′ biotinylated ME-inhibiting RNAi molecule of one of SEQ ID NOS: 10-12. The ME-inhibiting aptamer and ME-inhibiting RNAi molecule may optionally be formed using modified nucleotides to enhance their half life. Biotinylation of these aptamers at their 3′ ends is known not to interfere with the activity of RNA aptamers (see Chu et al., “Aptamer Mediated siRNA Delivery,” Nucl Acids Res. 34(10):e73 (2006), which is hereby incorporated by reference in its entirety).

The aptamers and RNAi will be synthesized by in vitro transcription from a double-stranded DNA template bearing a T7 RNA polymerase promoter. The aptamers will be purified using polyacrylamide gel electrophoresis, followed by overnight elution in water and ethanol precipitation.

Biotinylation of the purified aptamers and RNAi will be carried out using the procedures of Chu et al., “Aptamer Mediated siRNA Delivery,” Nucl Acids Res. 34(10):e73 (2006), which is hereby incorporated by reference in its entirety. Briefly, purified aptamer (150 nM) will be oxidized in 100 mM NaOAc (pH 5.0), 100 mM NaIO₄ (90 min, RT, dark), and the oxidized aptamers will be recovered via ethanol precipitation. The oxidized RNA will be reacted with 200 pmol of freshly prepared biotin-hydrazide in 500 μL 100 mM NaOAc (pH 5.0) (3 h, RT), and the hydrazide removed. The biotinylated RNA will be gel-purified.

Using 100 mM KOAc, 30 mM HEPES-KOH (pH 7.4) and 2 mM MgOAc buffer, 200 pmol of each biotinylated aptamer (1:1 ratio) will be introduced with 100 pmol of streptavidin. The complex will be allowed to equilibrate for a minimum of 10 minutes and then be stored on ice until its use.

AML-specific aptamer KH1C12 conjugates will be added directly to cell culture media (500 μl) at a final concentration of either 10 nM, 50 nM, or 100 nM conjugate. AML positive cell lines will be introduced at cell densities of 10⁴-10⁶, and cells will be assessed at 24, 48, and 72 hours after the addition of the conjugate for cell survival.

With positive in vitro results, these same cell lines and conjugates will be used in a xenograft model of human AML as described in the preceding Examples or Zuber et al., “Mouse Models of Human AML Accurately Predict Chemotherapy Response,” Genes Dev. 23:877-889 (2009), which is hereby incorporated by reference in its entirety (see also Wunderlich et al., “AML Cells Are Differentially Sensitive to Chemotherapy Treatment in a Human Xenograft Model,” Blood 121(12):e90-7 (2013), which is hereby incorporated by reference in its entirety). Mice bearing AML positive xenografts will be used. Seven days following implantation animals in several test groups will be administered 500 μg/kg, 2.5 mg/kg, or 12.5 mg/kg of the conjugate via i.p. injection every three days Animals will be maintained for 20 weeks following start of treatment. Tumor weights will be assessed every three days beginning the day treatment is started. Assessment of treatment versus control will be measured by percent test/control (% T/C) tumor weights calculated on each day that tumors are measured, tumor growth delay, and/or tumor regression.

### Prophetic Example 7

### Construction and Use of Conjugate for Targeting Expression Vector Delivery to AML Cells

A plasmid vector encoding an RNAi molecule of SEQ ID NOS: 6-11 will be used to prepare a polycation conjugated vectors. This will be carried out using the procedure of Moffatt et al., “Successful in vivo Tumor Targeting of Prostate-specific Membrane Antigen with a Highly Efficient J591/PEI/DNA Molecular Conjugate,” Gene Therapy 13:761-772 (2006), which is hereby incorporated by reference in its entirety, except that the plasmid vector will replace the β-gal plasmid of Moffatt and the prostate-specific mAb J591 will be replaced by an antibody that is specific for a AML cell surface marker (e.g., CD-33, CD-19, CD-20).

The addition of polyethylene glycol (PEG) (Sigma, Mr=3000) as a spacer between phenyl(di)boronic acid (PDBA) and the cancer cell surface marker will be carried out in a stepwise manner by first generating PDBA-PEG. About 300 mg of ω-amino-α-carboxyl PEG will be suspended in 0.1 M NaHCO₃ to a final concentration of 2 mg/ml. A 10 mM stock of PDBA-x-N-hydroxysuccinimide (NHS) will be made in N,N-dimethylformyl amine A molar ratio of 5:1 of PDBA to PEG will be produced by adding 4.4 ml of PDBA-x-NHS solution to the PEG solution. The sample will be dialyzed using a 1000-Mw membrane for 48 h at 41° C. against 20 mM HEPES buffer in the cold to yield PDBA-PEG. For PDBA-PEG-cancer cell surface marker formation, the cancer cell surface marker will be warmed in 37° C. for 5 minutes to activate the antibody, and then 300 mg of the antibody will be reacted with 0.1 M NaHCO₃ in a final volume of 250 ml. Following this, 1.3 ml of 0.1 M DTT solution will be added for 10 minutes in a 37° C. water bath to reduce disulfide bond formation. The DTT will be removed with Ultrafree-MC filter unit. In all, 300 mg of the 5:1 PDBA:PEG ratio will be introduced in a solution of 0.1M 2-[N-morpholino]ethane-sulfonic acid, pH 6.0, and 0.5 M NaCl, and the final volume will be brought up to 300 ml with distilled water. To activate the PEG for coupling to the cancer cell surface marker, 1-ethyl-3-(3-dimethyl-aminopropyl) carbodiimide and NHS will be added to final concentrations of 2 and 5 mM, respectively, allowed to react at room temperature for 15 minutes, and the reaction terminated with β-mercaptoethanol at a final concentration of 20 mM. Finally, 150 mg of the cancer cell surface marker solution (1 mg/ml) will be combined with the PDBA-PEG solution and allowed to proceed for 2 hours at room temperature, after which dialysis will be performed for 48 hours with 6000-8000 Mw cutoff membrane.

A working solution of 10 mM salicylhydroxamic acid-polyethylenimine (SHA-PEI) (pH 7.4) will be prepared in endotoxin-free water (BioWhittaker) and stored at 41° C. 4.5 ml of SHA-PEI will be added to 5.5 ml of 20 mM HEPES buffer, and then plasmid (6 mg) will be pipetted into polystyrene tubes and the solution brought up to 60 ml with HEPES buffer. Following this, 10 ml of the SHA-PEI solution will be added directly to the plasmid DNA solution and the solution allowed to incubate at room temperature for 5 minutes. After this, appropriate amounts of PDBA-PEG-cancer cell surface marker will be added and the incubation continued for an additional 5 minutes, which will result in the formation of the cancer cell surface maker/PEG/PEI/DNA(RNAi) vector.

This conjugate will be screened in vitro using the cell lines described in the preceding Examples, and also screened for in vivo activity using the xenograft mouse model of human AML described in the preceding Examples (Zuber et al., “Mouse Models of Human AML Accurately Predict Chemotherapy Response,” Genes Dev. 23:877-889 (2009) and Wunderlich et al., “AML Cells Are Differentially Sensitive to Chemotherapy Treatment in a Human Xenograft Model,” Blood 121(12):e90-7 (2013), both of which are hereby incorporated by reference in their entirety). Tetracycline will be administered to induce RNAi expression by the plasmid vector.

Mice bearing AML positive xenografts will be used. Seven days following implantation animals in several test groups will be administered 500 μg/kg, 2.5 mg/kg, or 12.5 mg/kg of the conjugate via i.p. injection every three days. Animals will be maintained for 20 weeks following start of treatment. Tumor weights will be assessed every three days beginning the day treatment is started. Assessment of treatment versus control will be measured by percent test/control (% T/C) tumor weights calculated on each day that tumors are measured, tumor growth delay, and/or tumor regression.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

1. A method of treating a cancerous condition mediated by the protein MDS1-EVI1 (“ME”), the method comprising:
administering to a patient an amount of an inhibitor of ME protein activity that is effective to cause cell death of cancer cells that are ME-dependent, thereby treating the cancerous condition.

2. The method according to claim 1, wherein the inhibitor of ME protein activity is an antibody or antibody fragment that binds specifically to a PR domain of the ME protein, an anti-ME nucleic acid aptamer, a dominant negative ME fragment, or a small molecule ME inhibitor.

3. The method according to claim 2, wherein the antibody or antibody fragment or the anti-ME aptamer binds specifically to at least a portion of the amino acid sequence of AGLGIWTKRKIEVGEKFGPYVGEQRSNLKDPSYG (amino acids 90-123 of SEQ ID NO: 1).

4. The method according to claim 1, wherein the inhibitor of ME activity is an inhibitory nucleic acid molecule that interferes with ME expression to cause a reduction in ME activity.

5. The method according to claim 4, wherein the inhibitory nucleic acid molecule is encoded by an expression vector.

6. The method according to claim 4, wherein the inhibitory nucleic acid molecule comprises an inhibitory RNA (“RNAi”) molecule.

7. The method according to claim 6, wherein the RNAi molecule targets nt 511-532 of GenBank sequence M21829, nt 3139-3167 of GenBank sequence M21829, nt 494-514 of the human ME-encoding sequence, nt 403-421 of the human ME-encoding sequence (uuCUGCAUAGAUGCCAGUCAAcc) (SEQ ID NO: 10), or nt 266-284 of the human ME-encoding sequence (ggGCAGGACUAGGAAUAUGGAcc) (SEQ ID NO: 11).

8. The method according to claim 1, wherein the cancerous condition is selected from the group of acute myeloid leukemia (AML), acute lymphoid leukemia (ALL), myelodysplastic syndrome, chronic myelogenous leukemia, and epithelial cancers where the 3q26.2 aberration is present.

9. The method according to claim 1, where the cancerous condition is characterized by the MLL-AF9 translocation t(9;11)(p22;q23) or MLL-ENL translocation t(11;19)(q23;p13.3).

10. The method according to claim 6, wherein the inhibitory nucleic acid molecule further comprises a nucleic acid aptamer targeting an myeloid leukemic cell, myelodysplastic cell, or an epithelial ovary, breast, head and neck, cervix, or lung cancer cell.

11. The method according to claim 10, wherein the aptamer is KH1C12 (SEQ ID NO: 12), which targets a myeloid leukemic cell.

12. The method according to claim 1, wherein said administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, by application to mucous membranes, or by introduction into one or more lymph nodes.

13. The method according to claim 1 further comprising administering an additional therapeutic agent selected from the group of a chemotherapeutic, radiation therapy, immunotherapy, and combinations thereof.

14. A method of causing cell death of a cancer cell that requires MDS1-EVI1 (“ME”) for survival, the method comprising:
introducing an inhibitor of ME activity into a cancer cell the requires ME for survival under conditions effective to cause cancer cell death.

15.-25. (canceled)

26. An inhibitor of ME protein activity selected from the group consisting of an antibody or antibody fragment that binds specifically to a PR domain of the ME protein, an anti-ME nucleic acid aptamer, a dominant negative ME fragment, or an inhibitory nucleic acid molecule that interferes with ME expression to cause a reduction in ME activity.

27. The inhibitor of ME protein activity according to claim 26, wherein the inhibitor of ME activity is an inhibitory nucleic acid molecule that interferes with ME expression to cause a reduction in ME activity.

28. The inhibitor of ME protein activity according to claim 27, wherein the inhibitory nucleic acid molecule comprises an inhibitory RNA (“RNAi”) molecule.

29. The inhibitor of ME protein activity according to claim 30, wherein the RNAi molecule targets nt 511-532 of GenBank sequence M21829, nt 3139-3167 of GenBank sequence M21829, nt 494-514 of the human ME-encoding sequence, nt 403-421 of the human ME-encoding sequence (uuCUGCAUAGAUGCCAGUCAAcc, SEQ ID NO: 10), or nt 266-284 of the human ME-encoding sequence (ggGCAGGACUAGGAAUAUGGAcc, SEQ ID NO: 114).

30. A pharmaceutical composition comprising an inhibitor of ME protein activity according to claim 26.


SEQUENCE LISTING (21 sequences)

SEQ ID NO: 1 - protein, 1239 aa, Homo sapiens
MRSKGRARKLATNNECVYGNYPEIPLEEMPDADGVASTPSLNIQEPCSPATSSEAFTPKEGSPYKAPIYIPDDIPIPAEFELRESNMPGAGLGIWTKRKIEVGEKFGPYVGEQRSNLKDPSYGWEILDEFYNVKFCIDASQPDVGSWLKYIRFAGCYDQHNLVACQINDQIFYRVVADIAPGEELLLFMKSEDYPHETMAPDIHEERQYRCEDCDQLFESKAELADHQKFPCSTPHSAFSMVEEDFQQKLESENDLQEIHTIQECKECDQVFPDLQSLEKHMLSHTEEREYKCDQCPKAFNWKSNLIRHQMSHDSGKHYECENCAKVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGANRHPAGLTFPTAPGFSFSFPGLFPSGLYHRPPLIPASSPVKGLSSTEQTNKSQSPLMTHPQILPATQDILKALSKHPSVGDNKPVELQPERSSEERPFEKISDQSESSDLDDVSTPSGSDLETTSGSDLESDIESDKEKFKENGKMFKDKVSPLQNLASINNKKEYSNHSIFSPSLEEQTAVSGAVNDSIKAIASIAEKYFGSTGLVGLQDKKVGALPYPSMFPLPFFPAFSQSMYPFPDRDLRSLPLKMEPQSPGEVKKLQKGSSESPFDLTTKRKDEKPLTPVPSKPPVTPATSQDQPLDLSMGSRSRASGTKLTEPRKNHVFGGKKGSNVESRPASDGSLQHARPTPFFMDPIYRVEKRKLTDPLEALKEKYLRPSPGFLFHPQFQLPDQRTWMSAIENMAEKLESFSALKPEASELLQSVPSMFNFRAPPNALPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESTGAILDDKEDAYFTEIRNFIGNSNHGSQSPRNVEERMNGSHFKDEKALVTSQNSDLLDDEEVEDEVLLDEEDEDNDITGKTGKEPVTSNLHEGNPEDDYEETSALEMSCKTSPVRYKEEEYKSGLSALDHIRHFTDSLKMRKMEDNQYSEAELSSFSTSHVPEELKQPLHRKSKSQAYAMMLSLSDKESLHSTSHSSSNVWHSMARAAAESSAIQSISHV

SEQ ID NO: 2 - DNA, 3720 bp, Homo sapiens
atgagatccaaaggcagggcaaggaaactggccacaaataatgagtgtgtatatggcaactaccctgaaatacctttggaagaaatgccagatgcagatggagtagccagcactccctccctcaatattcaagagccatgctctcctgccacatccagtgaagcattcactccaaaggagggttctccttacaaagcccccatctacatccctgatgatatccccattcctgctgagtttgaacttcgagagtcaaatatgcctggggcaggactaggaatatggaccaaaaggaagatcgaagtaggtgaaaagtttgggccttatgtgggagagcagaggtcaaacctgaaagaccccagttatggatgggagatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatga

SEQ ID NO: 3 - protein, 1232 aa, Mus musculus
MRSKGRARKLATSNECAYGNYPEIPLEEMPDADADGITSVPSLHIQEPCSPATSSESFTPKEGSPYKAPIYIPDDIPIPDEFELRESTMPGAGLGIWTKRKIEIGEKFGPYMGEQRSDLKDSSYGWEILDEFCNVKFCIDASQPDVGSWLKYIRFAGCYDQHNLVACQINDQIFYRVVADIAPGEELLLFMKSEEDPHEPMAPDIHEERQHRCEDCDQLFESKAELADHQKFPCSTPHSAFSMVEEDLQQNLESESDLREIHGNQDCKECDRVFPDLQSLEKHMLSHTEEREYKCDQCPKAFNWKSNLIRHQMSHDSGKHYECENCAKVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGTNRHPAGLTFPTAPGFSFSFPGLFPSGLYHRPPLIPASPPVKGLSSTEQSNKCQSPLLTHPQILPATQDILKALSKHPPVGDNKPVELLPERSSEERPLEKISDQSESSDLDDVSTPSGSDLETTSGSDLESDLESDKEKCKENGKMFKDKVSPLQNLASITNKKEHNNHSVFSASVEEQSAVSGAVNDSIKAIASIAEKYFGSTGLVGLQDKKVGALPYPSMFPLPFFPAFSQSMYPFPDRDLRSLPLKMEPQSPSEVKKLQKGSSESPFDLTTKRKDEKPLTSGPSKPSGTPATSQDQPLDLSMGSRGRASGTKLTEPRKNHVFGEKKGSNMDTRPSSDGSLQHARPTPFFMDPIYRVEKRKLTDPLEALKEKYLRPSPGFLFHPQMSAIENMAEKLESFSALKPEASELLQSVPSMFSFRAPPNTLPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESAGAILDDKEDAYFTEIRNFIGNSNHGSQSPRNMEERMNGSHFKDKKALATSQNSDLLDDEEVEDEVLLDEEDEDNDIPGKPRKELGVTRLDEEIPEDDYEEAGALEMSCKASPVRYKEEDYKSGLSALDHIRHFTDSLKMREMEENQYTDAELSSISSSHVPEELKQTLHRKSKSQAYAMMLSLSDKDSLHPTSHSSSNVWHSMARAAAESSAIQSISHV

SEQ ID NO: 4 - DNA, 3699 bp, Mus musculus
atgagatccaaaggcagggcaaggaaactggccacaagtaatgagtgtgcctatggcaactatcctgaaatacctttggaagaaatgccagatgctgatgcagatgggataaccagtgttccctccctccacattcaagagccatgctctcctgcgacgtccagtgagtcatttactcctaaggagggctcgccatacaaagctcccatctacatccctgatgacatccctattcctgatgagtttgagcttcgagagtcaactatgcctggagcaggacttggaatatggaccaaaaggaagattgaaataggcgaaaagtttgggccatacatgggagagcagagatcagacctgaaagattccagctatggatgggagatcttagatgagttttgcaatgtgaagttctgcatagatgccagtcaaccagatgtaggaagctggctcaagtacatcagattcgctggctgctatgatcagcacaaccttgttgcatgccagataaatgatcagatattctaccgagtagtcgcagacattgcgcctggggaagagctcttgctgttcatgaagagtgaagaggacccgcacgaacccatggcgcctgacatccacgaagaacggcagcaccgctgtgaggactgtgaccagctctttgaatccaaggcagagctagccgatcaccagaagttcccatgcagcacacctcactcggccttctccatggtggaggaggacttgcaacaaaacctggagagtgagagcgatctccgagagatccatggcaaccaggactgtaaggaatgtgaccgagttttccccgatctgcaaagcttggagaagcacatgctgtcacatactgaggagagggaatacaagtgtgatcagtgtcccaaggcatttaactggaagtccaatttaattcgccaccagatgtcacatgacagtggaaagcactatgagtgtgaaaactgtgccaaggttttcacggaccctagcaaccttcagcgacacattcgatctcagcatgttggtgcccgggctcatgcttgccccgagtgtggtaaaacatttgccacttcgtcaggcctcaaacagcacaagcacatccacagcagtgtgaagccctttatctgtgaggtctgccataaatcctatactcagttttcaaacctttgtcgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggcctggctgactattttggcaccaataggcatcctgctggtcttacctttccaacagctcctggattttcctttagctttcctggtctgtttccttcgggcttataccacaggcctcctttgatacccgctagtcctcctgttaaaggattatcaagtaccgaacagtcaaacaaatgtcaaagtcccctcttgacacatcctcagatactgccagctacacaggatattttgaaggcactatctaaacacccacctgtaggggacaataagccagtggaactgctgcccgagaggtcctctgaagagaggccccttgagaaaatcagtgaccagtcagagagtagtgaccttgatgatgtcagtacaccaagtggcagtgacctggaaacaacctcgggctctgatctggaaagtgaccttgaaagtgataaagagaaatgtaaagaaaatggtaaaatgttcaaagacaaagtaagccctctgcagaatctggcttcgataactaataagaaagaacacaacaatcattccgttttctcagcatctgtagaggagcaaagtgccgtgtcaggagctgtgaatgattctataaaggctattgcgtctattgctgaaaaatactttggttctacaggactggtggggctgcaagacaaaaaagttggagcgttaccttacccttccatgtttcccctcccgttttttccagcattctctcaatcaatgtacccatttcctgatagagacttgaggtcgttacctttgaaaatggagccccaatcaccaagtgaagttaagaaactgcagaagggaagctctgagtccccttttgacctcaccactaagagaaaggatgagaagcccttgacttcaggcccctcgaagccttcaggaacaccagccacaagccaagaccagcccctggatctaagtatgggcagtaggggtagagccagtgggacaaagttgactgagcctcgaaaaaaccatgtgtttggggaaaagaaaggaagcaacatggatactaggccatcttcagatggctccttgcagcatgccagacccactcccttcttcatggaccccatttatagagtagagaaaagaaagttaactgacccgcttgaagctttgaaagaaaaatacttgagaccttctccaggattcttgtttcacccgcaaatgtcagcaattgagaacatggcagaaaagctggaaagcttcagcgccctcaaacctgaggccagcgagctcctgcagtccgtgccctccatgttcagcttccgagctcctcccaacaccctgccagagaacctgctgcggaaggggaaagagcgctacacctgcaggtactgtggcaagatatttccaaggtctgcgaacctaacacggcacttgagaacccacacaggagagcaaccttacagatgcaaatactgtgatagatcattcagcatttcttccaacctgcagcgacatgtgcgcaacatccacaacaaggagaagccatttaagtgtcatttatgtgacagatgttttggtcaacaaaccaatcttgacagacacctgaagaaacatgagaacggcaacatgtctgggacggcaacgtcctcgcctcactcagagctagaaagcgcaggcgcaatcctggatgacaaagaagatgcgtactttacagagatccgcaatttcatcgggaacagcaaccatggtagccagtctcctcggaacatggaagagaggatgaatggcagtcacttcaaggataaaaaggctttggcaaccagccaaaattcagatttattggatgatgaagaagtagaagatgaggtgttgttggatgaggaggatgaagacaatgatattcctggaaagcccagaaaggagctaggggtgactcgtttagacgaggagatcccggaggatgactacgaagaagctggtgccctggagatgagctgtaaggcgtccccggtgaggtataaagaggaagactataaatctggcctttctgctctagatcacataaggcacttcacagatagcctcaaaatgagggaaatggaagagaatcaatacactgacgctgagctgtcctccattagttcttctcatgtgccagaggagcttaaacagacgttacacagaaagtccaaatcacaggcatatgctatgatgttgtcactgtctgacaaggattccctccatcccacctcccacagttcctccaacgtgtggcacagcatggcaagggctgcagcagaatccagtgccatccagtccataagccatgtatga

SEQ ID NO: 5 - protein, 110 aa, Artificial
  ME Consensus sequence
LRESXMPGAGLGIWTKRKIEXGEKFGPYXGEQRSXLKDXSYGWEILDEFXNVKFCIDASQPDVGSWLKYIRFAGCYDQHNLVACQINDQIFYRVVADIAPGEELLLFMKS

SEQ ID NO: 6 - DNA, 72 bp, Artificial
  Sequence encoding shRNA designated sh11
gatccgagcgcctgacatccacgaagaacttcaagagagttcttcgtggatgtcaggcgctttttacgcgtg

SEQ ID NO: 7 - DNA, 72 bp, Artificial
  Sequence encoding shRNA designated sh54
gatccgagttgttggatgaggaggatgaattcaagagattcatcctcctcatccaacaactttttacgcgtg

SEQ ID NO: 8 - DNA, 26 bp, Artificial
  Sequence encoding shRNA targeting murine ME PR domain
cctattcctgatgagtttgagcttca

SEQ ID NO: 9 - DNA, 21 bp, Artificial
  Sequence encoding SiRNA targeting human ME PR domain
gccagataaatgatcagatat

SEQ ID NO: 10 - RNA, 23 nt, Artificial
  RNAi targeting human ME
uucugcauagaugccagucaacc

SEQ ID NO: 11 - RNA, 23 nt, Artificial
  RNAi targeting human ME
gggcaggacuaggaauauggacc

SEQ ID NO: 12 - DNA, 78 bp, Artificial
  Aptamer
atccagagtgacgcagcatgccctagttactactactctttttagcaaacgccctcgctttggacacggtggcttagt

SEQ ID NO: 13 - DNA, 30 bp, Artificial
  PCR primer
atgaattcgcatgagatccaaaggcagggc

SEQ ID NO: 14 - DNA, 34 bp, Artificial
  PCR primer
atgaattcttcacctggtctcccatccatagctg

SEQ ID NO: 15 - protein, 127 aa, Mus musculus
DDIPIPDEFELRESTMPGAGLGIWTKRKIEVGEKFGPYVGEQRSNLKDPSYGWEILDEFCNVKFCIDASQPDVGSWLKYIRFAGCYDQHNLVACQINDQIFYRVVADIAPGEELLLFMKSEEDYPHE

SEQ ID NO: 16 - protein, 112 aa, Homo sapiens
YDLCIFRTDDGRGWGVRTLEKIRKNSFVMEYVGEIITSEEAERRDYVEDVYTVDAAYYGNISHFVNHSCDPNLQVYNVFIDNLDERLPRIAFFATRTIRAGEELTFDYNMQV

SEQ ID NO: 17 - protein, 114 aa, P. sativa
EEGVITAKTPVKASVVTEGLGLVALKDISRNDVILQVPKRLWINRAFSRLRNENLVVVPMADLINHSAGVTTEDHAYEVKGAAGLFSWDYLFSLKSPLSVKAGEQVYIQYDLNK

SEQ ID NO: 18 - protein, 33 aa, Homo sapiens
AGWGIFIKDPVKNEFISEYCGEIISQDEADRRG

SEQ ID NO: 19 - protein, 34 aa, Mus musculus
AGWGTFIKESVQKNEFISEYCGELISQDEADRRG

SEQ ID NO: 20 - protein, 34 aa, Drosophila melanogaster
AGWGIFLKEGAQKNEFISEYCGEIISQDEADRRG

SEQ ID NO: 21 - protein, 34 aa, Homo sapiens
HGRGLFCKRNIDAGEMVIEYAGNVIRSIQTDKRE